(19) United States
(12) Patent Application Publication
(10) Pub. No.: US 2010/0273772 A1
(43) Pub. Date: Oct. 28, 2010

(54) Bisaryl Alkynylamides as Negative Allosteric Modulators of Metabotropic Glutamate Receptor 5 (MGLUR5)

(75) Inventors: Steven Victor O'Neil, Clinton, CT (US); Benjamin Miller Zegarelli, West Hartford, CT (US); Dane Mark Springer, Yardley, PA (US); David Zenan Li, Princeton, NJ (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/765,068

(22) Filed: Apr. 22, 2010

Related U.S. Application Data
(60) Provisional application No. 61/171,878, filed on Apr. 23, 2009.

(51) Int. Cl. A61K 31/553; C07D 401/10; A61K 31/4439; A61K 31/4725; C07D 487/04; A61K 31/4985; C07D 267/14; C07D 243/14; A61K 31/5513; C07D 223/16; A61K 31/55; C07D 471/04; A61K 31/4375; C07D 209/44; A61K 31/4035; A61P 25/00; A61P 29/00

(52) U.S. Cl. 514/211.09; 546/277.1; 514/339; 546/146; 514/307; 546/143; 514/310; 544/350; 514/249; 540/552; 540/567; 514/221; 540/594; 514/213.01; 546/122; 514/300; 548/482; 514/416

(57) ABSTRACT

Disclosed are compounds of Formula I:

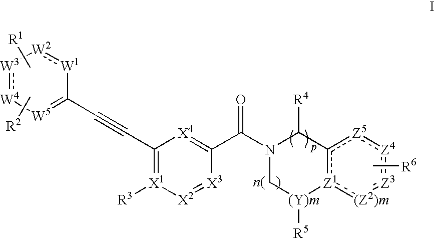

pharmaceutical compositions containing compounds of Formula I, and the use of compounds of Formula I to treat diseases and disorders including schizophrenia, paranoia, depression, manic-depressive illness and anxiety wherein W¹-W⁵, X¹-X⁴, Y, Z¹-Z⁵, m, n, p, and R¹-R⁶ in Formula I are defined in the specification.

### CROSS-REFERENCE TO RELATED APPLICATIONS

This application claims the benefit under 35 USC 119(e) of U.S. provisional application 61/171,878 filed on Apr. 23, 2009, which is incorporated herein by reference in their entirety.

### FIELD

In one aspect, this invention relates to bisaryl alkynylamides as negative allosteric modulators of metabotropic glutamate receptor 5 (mGluR5), and methods for their preparation. In a further aspect, the invention provides methods for using the mGluR5 antagonists for treatment of diseases and disorders including schizophrenia, paranoia, depression, bipolar disorder, anxiety, pain, and addiction.

### BACKGROUND

The metabotropic glutamate 5 receptor (mGluR5) is a G-protein-coupled metabotropic glutamate receptor that plays a role as a modulator of synaptic plasticity, ion channel activity, and excitotoxicity (Bach et al., Metabotropic Glutamate Receptor 5 Modulators and their Potential Therapeutic Applications, Department of Med. Chemistry, AstraZeneca R and D Moelndal, Moelndal, Sweden, Expert Opinion on Therapeutic Patents 2007, 17(4), 371-384 and references therein).

Recent evidence indicates that current mGluR5 antagonists are not sufficiently selective, and cause off-target effects, such as inhibition of NMDA receptors. Thus, there exists an ongoing need for compounds that more selectively bind to mGluR5, and that are useful in repressing and/or treating disorders such as schizophrenia, paranoia, depression, manic-depressive illness, anxiety, pain, and addiction.

Non-competitive mGluR ligands have been identified which bind within the receptor transmembrane heptahelical domain. These include negative allosteric modulators. Negative allosteric modulators include both non-competitive antagonists and inverse agonists. Negative allosteric modulation offers the potential for improved selectivity, particularly for individual receptors within the mGluR family, and enhanced chemical tractability relative to competitive agonists/antagonists. This invention is directed to these, as well as other, important ends.

### SUMMARY

In one aspect, the invention provides compounds of Formula I:

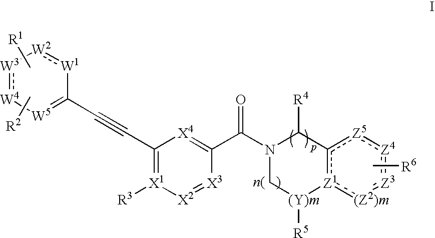

or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

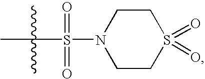

the C₁-C₆alkyl, C₃-C₆cycloalkyl, aryl, heteroaryl, aryloxy, heteroaryloxy, C₁-C₆alkylNR⁷R⁸ and

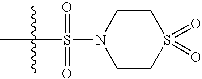

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S.

In another aspect, the invention provides pharmaceutical compositions containing a compound of the invention or a pharmaceutically acceptable salt thereof, and a pharmaceutically acceptable carrier.

In a further aspect, the invention provides methods for the treatment of a patient suffering from depression, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia or obesity.

In yet another aspect, the invention provides methods for producing compounds of Formula I.

Other aspects of the present teachings are described further in the following

### DETAILED DESCRIPTION

In accordance with the invention, there are provided compounds of Formula I:

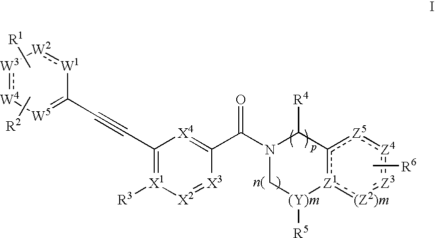

or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

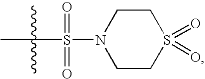

the C₁-C₆alkyl, C₃-C₆cycloalkyl, aryl, heteroaryl, aryloxy, heteroaryloxy, C₁-C₆alkylNR⁷R⁸ and

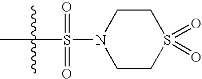

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S.

In some embodiments of Formula I, W¹ is selected from N and C. In certain embodiments, W¹ is N. In yet further embodiments, Z⁵ is N. In some further embodiments, Z⁴ is N.

In some embodiments of Formula I, R³ is selected from —O(C₁-C₆alkyl), halogen, halo-C₁-C₆alkyl, and halo-C₁-C₆alkoxy. In certain of these embodiments, R³ is selected from methoxy, ethoxy, —OCF₃, and —OCHF₂.

In some embodiments of Formula I, n is 1. In some embodiments m is 0.

In some embodiments of Formula I, each of R¹, R², R⁴, R⁵, R⁶, R⁷, and R⁸ is H. In certain other embodiments of Formula I, each of W², W³, W⁴, W⁵, X¹, X², X³, X⁴, Z¹, Z², Z³, and Z⁴ is C.

In some embodiments of Formula I, R⁵ and R⁶ are each H.

In some embodiments of Formula I, the compounds include
2-[4-methoxy-3-(pyridin-2-ylethynyl)benzoyl]isoindoline;
2-[4-methoxy-3-(pyridin-2-ylethynyl)benzoyl]-1,2,3,4-tetrahydroisoquinoline;
5-fluoro-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-5-(trifluoromethyl)-1,2,3,4-tetrahydroisoquinoline;
2-[4-methoxy-3-(pyridin-2-ylethynyl)benzoyl]-8-(trifluoromethyl)-1,2,3,4-tetrahydroisoquinoline;
5,8-difluoro-2-[4-methoxy-3-(pyridin-2-ylethynyl)benzoyl]-1,2,3,4-tetrahydroisoquinoline;
5,7-dichloro-2-[4-methoxy-3-(pyridin-2-ylethynyl)benzoyl]-1,2,3,4-tetrahydroisoquinoline;
7-chloro-2-[4-methoxy-3-(pyridin-2-ylethynyl)benzoyl]-1,2,3,4-tetrahydroisoquinoline;
7-fluoro-2-[4-methoxy-3-(pyridin-2-ylethynyl)benzoyl]-1,2,3,4-tetrahydroisoquinoline;
5-chloro-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1,2,3,4-tetrahydroisoquinoline-7-carbonitrile;
2-[4-methoxy-3-(pyridin-2-ylethynyl)benzoyl]-2,3,4,5-tetrahydro-1H-pyrido[4,3-b]indole;
5-[4-methoxy-3-(pyridin-2-ylethynyl)benzoyl]-4,5,6,7-tetrahydrothieno[3,2-c]pyridine;
3-bromo-6-[4-methoxy-3-(pyridin-2-ylethynyl)benzoyl]-5,6,7,8-tetrahydro-1,6-naphthyridine;
6-[4-methoxy-3-(pyridin-2-ylethynyl)benzoyl]-5,6,7,8-tetrahydropyrido[4,3-d]pyrimidin-4(3H)-one;
2-[4-methoxy-3-(pyridin-2-ylethynyl)benzoyl]-7-(trifluoromethyl)-1,2,3,4-tetrahydroisoquinoline;
1-{2-[4-methoxy-3-(pyridin-2-ylethynyl)benzoyl]-2,3-dihydro-1H-isoindol-1-yl}-2-methylpropan-2-ol;
9-[4-methoxy-3-(pyridin-2-ylethynyl)benzoyl]-1,2,3,4-tetrahydro-1,4-epiminonaphthalene;
5,8-dimethoxy-2-[4-methoxy-3-(pyridin-2-ylethynyl)benzoyl]-1,2,3,4-tetrahydroisoquinoline;
6,7-dimethoxy-2-[4-methoxy-3-(pyridin-2-ylethynyl)benzoyl]-1,2,3,4-tetrahydroisoquinoline;
8-methoxy-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1-(2-thienyl)-1,2,3,4-tetrahydroisoquinoline;
2-[4-methoxy-3-(pyridin-2-ylethynyl)benzoyl]-8-[1-(methylsulfonyl)piperidin-4-yl]-1,2,3,4-tetrahydroisoquinoline;
2-[4-methoxy-3-(pyridin-2-ylethynyl)benzoyl]-4-[4-(trifluoromethyl)phenoxy]-1,2,3,4-tetrahydroisoquinoline;
4-[(1,1-dioxidothiomorpholin-4-yl)sulfonyl]-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1-phenyl-1,2,3,4-tetrahydroisoquinoline;
1-(2-furyl)-2-[4-methoxy-3-(pyridin-2-ylethynyl)benzoyl]-1,2,3,4-tetrahydroisoquinoline;
(1S)-2-[4-methoxy-3-(pyridin-2-ylethynyl)benzoyl]-1-[(2S)-tetrahydrofuran-2-yl]-1,2,3,4-tetrahydroisoquinoline;
N-({2-[4-methoxy-3-(pyridin-2-ylethynyl)benzoyl]-1,2,3,4-tetrahydroisoquinolin-4-yl}methyl)aniline;
1-benzyl-2-[4-methoxy-3-(pyridin-2-ylethynyl)benzoyl]-1,2,3,4-tetrahydroisoquinoline; 6-methoxy-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-5-methylisoindoline; 5-bromo-2-[4-methoxy-3-(pyridin-2-ylethynyl)benzoyl]isoindoline;
5,6-dichloro-2-[4-methoxy-3-(pyridin-2-ylethynyl)benzoyl]isoindoline; 2-[4-methoxy-3-(pyridin-2-ylethynyl)benzoyl]-5-nitroisoindoline;
4-fluoro-2-[4-methoxy-3-(pyridin-2-ylethynyl)benzoyl]isoindoline; 5-fluoro-2-[4-methoxy-3-(pyridin-2-ylethynyl)benzoyl]isoindoline;
4-chloro-2-[4-methoxy-3-(pyridin-2-ylethynyl)benzoyl]isoindoline;
6-[4-methoxy-3-(pyridin-2-ylethynyl)benzoyl]-5,6,7,8-tetrahydro-1,6-naphthyridine; 6-[4-methoxy-3-(pyridin-2-ylethynyl)benzoyl]-3-methyl-5,6,7,8-tetrahydro-1,6-naphthyridine;
2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-6,7-dimethyl-2,3-dihydro-1H-pyrrolo[3,4-c]pyridine;
7-chloro-2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-6-methyl-2,3-dihydro-1H-pyrrolo[3,4-c]pyridin-7-amine;
7-methoxy-2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-2,3-dihydro-1H-pyrrolo[3,4-c]pyridine;
5-{[4-methoxy-3-(pyridin-2-ylethynyl)phenyl]carbonyl}-2-methyl-4,5,6,7-tetrahydro[1,3]oxazolo[4,5-c]pyridine;
5-{[4-methoxy-3-(pyridin-2-ylethynyl)phenyl]carbonyl}-2-methyl-4,5,6,7-tetrahydro[1,3]thiazolo[5,4-c]pyridine;
7-{[4-methoxy-3-(pyridin-2-ylethynyl)phenyl]carbonyl}-3-(trifluoromethyl)-5,6,7,8-tetrahydro[1,2,4]triazolo[4,3-a]pyrazine;
7-{[4-methoxy-3-(pyridin-2-ylethynyl)phenyl]carbonyl}-5,6,7,8-tetrahydro-1,7-naphthyridine;
3-chloro-6-{[4-methoxy-3-(pyridin-2-ylethynyl)phenyl]carbonyl}-5,6,7,8-tetrahydro-1,6-naphthyridine;
6-{[4-methoxy-3-(pyridin-2-ylethynyl)phenyl]carbonyl}-3-(trifluoromethyl)-5,6,7,8-tetrahydro-1,6-naphthyridine;
5-{[4-methoxy-3-(pyridin-2-ylethynyl)phenyl]carbonyl}-4,5,6,7-tetrahydro-1H-pyrazolo[4,3-c]pyridine;
6-{[4-methoxy-3-(pyridin-2-ylethynyl)phenyl]carbonyl}-6,7-dihydro-5H-pyrrolo[3,4-b]pyridine;
4-{[4-methoxy-3-(pyridin-2-ylethynyl)phenyl]carbonyl}-2,3,4,5-tetrahydro-1,4-benzoxazepine;
4-{[4-methoxy-3-(pyridin-2-ylethynyl)phenyl]carbonyl}-2,3,4,5-tetrahydro-1H-1,4-benzodiazepine;
3-{[4-methoxy-3-(pyridin-2-ylethynyl)phenyl]carbonyl}-2,3,4,5-tetrahydro-1H-3-benzazepine;
5-{[4-methoxy-3-(pyridin-2-ylethynyl)phenyl]carbonyl}-4,5,6,7-tetrahydro-1H-imidazo[4,5-c]pyridine;
2-{[4-methoxy-3-(pyridin-2-ylethynyl)phenyl]carbonyl}-2,3,4,5-tetrahydro-1H-2-benzazepine;
2-[4-chloro-3-(pyridin-2-ylethynyl)benzoyl]isoindoline; 2-[4-chloro-3-(pyridin-2-ylethynyl)benzoyl]-1,2,3,4-tetrahydroisoquinoline;
7-{[4-chloro-3-(pyridin-2-ylethynyl)phenyl]carbonyl}-5,6,7,8-tetrahydro-1,7-naphthyridine;
2-{[4-chloro-3-(pyridin-2-ylethynyl)phenyl]carbonyl}-5,8-difluoro-1,2,3,4-tetrahydroisoquinoline;
2-{[4-chloro-3-(pyridin-2-ylethynyl)phenyl]carbonyl}-5-fluoro-1,2,3,4-tetrahydroisoquinoline;
6-{[4-chloro-3-(pyridin-2-ylethynyl)phenyl]carbonyl}-6,7-dihydro-5H-pyrrolo[3,4-b]pyridine;
5-{[4-chloro-3-(pyridin-2-ylethynyl)phenyl]carbonyl}-4,5,6,7-tetrahydro-1H-pyrazolo[4,3-c]pyridine;
5-{[4-chloro-3-(pyridin-2-ylethynyl)phenyl]carbonyl}-4,5,6,7-tetrahydro-1H-imidazo[4,5-c]pyridine;
6-{[4-chloro-3-(pyridin-2-ylethynyl)phenyl]carbonyl}-5,6,7,8-tetrahydro-1,6-naphthyridine;
5-chloro-2-{[4-chloro-3-(pyridin-2-ylethynyl)phenyl]carbonyl}-1,2,3,4-tetrahydroisoquinoline;
2-{[4-chloro-3-(pyridin-2-ylethynyl)phenyl]carbonyl}-7-fluoro-1,2,3,4-tetrahydroisoquinoline;
2-[4-fluoro-3-(pyridin-2-ylethynyl)benzoyl]-1,2,3,4-tetrahydroisoquinoline;
7-chloro-2-4-fluoro-3-(pyridin-2-ylethynyl)benzoyl]-1,2,3,4-tetrahydroisoquinoline;
5-chloro-2-[4-fluoro-3-(pyridin-2-ylethynyl)benzoyl]-1,2,3,4-tetrahydroisoquinoline;
2-[4-fluoro-3-(pyridin-2-ylethynyl)benzoyl]isoindoline;
2-[4-ethoxy-3-(pyridin-2-ylethynyl)benzoyl]-1,2,3,4-tetrahydroisoquinoline;
7-chloro-2-[4-ethoxy-3-(pyridin-2-ylethynyl)benzoyl]-1,2,3,4-tetrahydroisoquinoline;
5-chloro-2-[4-ethoxy-3-(pyridin-2-ylethynyl)benzoyl]-1,2,3,4-tetrahydroisoquinoline;
2-[4-ethoxy-3-(pyridin-2-ylethynyl)benzoyl]isoindoline;
2-{[4-(cyclopropylmethoxy)-3-(pyridin-2-ylethynyl)phenyl]carbonyl}-1,2,3,4-tetrahydroisoquinoline;
2-{[4-(cyclopropylmethoxy)-3-(pyridin-2-ylethynyl)phenyl]carbonyl}-2,3-dihydro-1H-isoindole;
2-[4-methyl-3-(pyridin-2-ylethynyl)benzoyl]isoindoline;
2-[4-methyl-3-(pyridin-2-ylethynyl)benzoyl]-1,2,3,4-tetrahydroisoquinoline;
7-chloro-2-[4-methyl-3-(pyridin-2-ylethynyl)benzoyl]-1,2,3,4-tetrahydroisoquinoline;
5-chloro-2-[4-methyl-3-(pyridin-2-ylethynyl)benzoyl]-1,2,3,4-tetrahydroisoquinoline;
6-{[4-methyl-3-(pyridin-2-ylethynyl)phenyl]carbonyl}-5,6,7,8-tetrahydro-1,6-naphthyridine;
7-{[4-methyl-3-(pyridin-2-ylethynyl)phenyl]carbonyl}-5,6,7,8-tetrahydro-1,7-naphthyridine;
5-{[4-methyl-3-(pyridin-2-ylethynyl)phenyl]carbonyl}-4,5,6,7-tetrahydro-3H-imidazo[4,5-c]pyridine;
5-{[4-methyl-3-(pyridin-2-ylethynyl)phenyl]carbonyl}-4,5,6,7-tetrahydro-1H-pyrazolo[4,3-c]pyridine;
2-methyl-5-{[4-methyl-3-(pyridin-2-ylethynyl)phenyl]carbonyl}-4,5,6,7-tetrahydro[1,3]thiazolo[5,4-c]pyridine;
2-methyl-5-{[4-methyl-3-(pyridin-2-ylethynyl)phenyl]carbonyl}-4,5,6,7-tetrahydro[1,3]oxazolo[4,5-c]pyridine;
6-{[4-methyl-3-(pyridin-2-ylethynyl)phenyl]carbonyl}-6,7-dihydro-5H-pyrrolo[3,4-b]pyridine;
6-{[3-(pyridin-2-ylethynyl)-4-(trifluoromethyl)phenyl]carbonyl}-6,7-dihydro-5H-pyrrolo[3,4-b]pyridine;
2-{[3-(pyridin-2-ylethynyl)-4-(trifluoromethyl)phenyl]carbonyl}-1,2,3,4-tetrahydroisoquinoline;
7-chloro-2-{[3-(pyridin-2-ylethynyl)-4-(trifluoromethyl)phenyl]carbonyl}-1,2,3,4-tetrahydroisoquinoline;
5-chloro-2-{[3-(pyridin-2-ylethynyl)-4-(trifluoromethyl)phenyl]carbonyl}-1,2,3,4-tetrahydroisoquinoline;
2-{[3-(pyridin-2-ylethynyl)-4-(trifluoromethyl)phenyl]carbonyl}-2,3-dihydro-1H-isoindole;
3-bromo-6-{[3-(pyridin-2-ylethynyl)-4-(trifluoromethyl)phenyl]carbonyl}-5,6,7,8-tetrahydro-1,6-naphthyridine;
6-{[3-(pyridin-2-ylethynyl)-4-(trifluoromethyl)phenyl]carbonyl}-5,6,7,8-tetrahydro-1,6-naphthyridine;
7-{[3-(pyridin-2-ylethynyl)-4-(trifluoromethyl)phenyl]carbonyl}-5,6,7,8-tetrahydro-1,7-naphthyridine;
5-{[3-(pyridin-2-ylethynyl)-4-(trifluoromethyl)phenyl]carbonyl}-4,5,6,7-tetrahydro-1H-pyrazolo[4,3-c]pyridine;
5-{[3-(pyridin-2-ylethynyl)-4-(trifluoromethyl)phenyl]carbonyl}-4,5,6,7-tetrahydro-1H-imidazo[4,5-c]pyridine;
2-{[3-(pyridin-2-ylethynyl)-4-(trifluoromethyl)phenyl]carbonyl}-2,3-dihydro-1H-pyrrolo[3,4-c]pyridine;
2-{4-methoxy-3-[(3-methoxyphenyl)ethynyl]benzoyl}isoindoline; 2-{3-[(2,4-difluorophenyl)ethynyl]-4-methoxybenzoyl}isoindoline;
2-{3-[(4-fluoro-3-methylphenyl)ethynyl]-4-methoxybenzoyl}isoindoline;
2-(4-methoxy-3-{[2-(trifluoromethyl)phenyl]ethynyl}benzoyl)isoindoline;
2-{4-methoxy-3-[(2-methoxyphenyl)ethynyl]benzoyl}isoindoline;
2-[3-(cyclohex-1-en-1-ylethynyl)-4-methoxybenzoyl]isoindoline; 2-[4-methoxy-3-(pyridin-3-ylethynyl)benzoyl]isoindoline;
2-{3-[(2-chlorophenyl)ethynyl]-4-methoxybenzoyl}isoindoline;
4-{[5-(1,3-dihydro-2H-isoindol-2-ylcarbonyl)-2-methoxyphenyl]ethynyl}benzonitrile;
3-{[5-(1,3-dihydro-2H-isoindol-2-ylcarbonyl)-2-methoxyphenyl]ethynyl}phenol;
2-{3-[(3,5-dimethoxyphenyl)ethynyl]-4-methoxybenzoyl}isoindoline;
2-[4-methoxy-3-(phenylethynyl)benzoyl]isoindoline;
2-{4-methoxy-3-[(3-methylphenyl)ethynyl]benzoyl}isoindoline;
2-{3-[(4-fluorophenyl)ethynyl]-4-methoxybenzoyl}isoindoline;
2-{4-methoxy-3-[(4-methoxyphenyl)ethynyl]benzoyl}isoindoline;
2-{3-[(3-chlorophenyl)ethynyl]-4-methoxybenzoyl}isoindoline; 2-[3-(Pyridin-2-ylethynyl)benzoyl]isoindoline;
(4-(difluoromethoxy)-3-(pyridin-2-ylethynyl)phenyl)(5H-pyrrolo[3,4-b]pyridin-6(7H)-yl)methanone;
2-{4-methoxy-3-[(6-methylpyridin-2-yl)ethynyl]benzoyl}isoindoline; and
2-{4-methoxy-3-[(6-methylpyridin-2-yl)ethynyl]benzoyl}-1,2,3,4-tetrahydroisoquinoline; or pharmaceutically acceptable salts thereof.

In yet another embodiment of Formula I, a pharmaceutical composition comprising a compound of Formula I as defined herein or a pharmaceutically acceptable salt thereof and a pharmaceutically acceptable excipient.

In yet another embodiment, a method of treating a subject suffering from depression, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia or obesity comprising administering to the subject in need of treatment a therapeutically effective amount of a compound of Formula I:

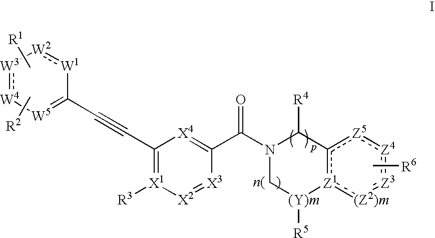

or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

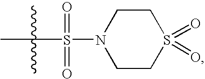

the C₁-C₆alkyl, C₃-C₆cycloalkyl, aryl, heteroaryl, aryloxy, heteroaryloxy, C₁-C₆alkylNR⁷R⁸ and

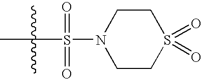

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S.

Another embodiment is the use of a compound of Formula I as defined herein or a pharmaceutically acceptable salt thereof, for the preparation of a medicament for the treatment of a disorder or condition selected from depression, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity.

Another embodiment includes combining one or more compounds of Formula I or pharmaceutically acceptable salts thereof and one or more pharmacologically active compounds which are different than the one or more compounds of Formula I.

Prodrugs of the compounds of Formula I are also embraced by the present invention. The term “prodrug”, as used herein, means a compound which is convertible in vivo by metabolic means (e.g. by hydrolysis) to a compound of formula I. Various forms of prodrugs are known in the art, for example, as discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs”, Textbook of Drug Design and Development, Chapter 5, 113-191 (1991), Bundgaard, et al., Journal of Drug Delivery reviews, 8:1-38 (1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is incorporated by reference in its entirety.

The mGluR5 antagonists disclosed herein are useful for treating diseases and disorders including depression, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity. Accordingly, in some embodiments, the invention provides pharmaceutical compositions comprising a pharmaceutically acceptable carrier and a compound of Formula I, or a pharmaceutically acceptable salt, hydrate or prodrug thereof. In further embodiments, the invention provides methods of treating a patient suffering from a condition such as depression, anxiety (including panic disorders, social anxiety, obsessive compulsive disorders, generalized anxiety disorders, phobias), post-traumatic stress disorder, is bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia or obesity comprising providing a therapeutically effective amount of compound of Formula I, or a pharmaceutically acceptable salt, hydrate, solvate or prodrug thereof.

Some compounds of the present invention can contain an asymmetric atom (also referred as a chiral center), and some of the compounds can contain one or more asymmetric atoms or centers, which can thus give rise to optical isomers (enantiomers) and diastereomers (geometric isomers). The present invention includes such optical isomers and diastereomers, as well as, the racemic and resolved, enantiomerically pure R and S stereoisomers, as well as, other mixtures of the R and S stereoisomers and pharmaceutically acceptable salts, hydrates, solvates, metabolites and prodrugs thereof. Optical isomers can be obtained in pure form by standard procedures known to those skilled in the art, and include, but are not limited to, chiral chromatography, diastereomeric salt formation, kinetic resolution, and asymmetric synthesis. The present teachings also encompass cis and trans or E/Z isomers of compounds containing alkenyl moieties (e.g., alkenes and imines). It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

As used herein, the term “alkyl” as a group or part of a group is intended to denote hydrocarbon groups including straight chain, branched and cyclic saturated hydrocarbons. Alkyl groups can contain 1-20, or 1-12, or 1-6 carbon atoms. The term “lower alkyl” is intended to mean an alkyl group having up to 6 carbon atoms. Nonlimiting examples of straight chain and branched alkyl groups include methyl (Me), ethyl (Et), propyl (e.g., n-propyl and isopropyl), butyl (e.g., n-butyl, isobutyl, s-butyl, and t-butyl), pentyl groups (e.g., n-pentyl, isopentyl, and neopentyl), hexyl groups, and the like.

The term “cycloalkyl” is intended to mean a monocyclic or bicyclic saturated hydrocarbon group having the indicated number of carbon atoms. For example, a C₃-C₈ cycloalkyl group would include cyclopropyl, cyclobutyl, cyclopentyl, cyclohexyl, cycloheptyl and cyclooctyl groups, as well as polycyclic systems (e.g., containing fused, bridged, and/or spiro ring systems). Any suitable ring position of a cyclic alkyl group can be covalently linked to the defined chemical structure. Unless otherwise indicated, alkyl groups are unsubstituted. However, where indicated, alkyl groups may be substituted with one or more independently selected substituents as described herein.

As used herein, “Aryl” and “arylene” refer to monovalent and divalent aromatic groups, respectively, including 5- and 6-membered monocyclic aromatic groups that contain 0 to 4 heteroatoms independently selected from nitrogen, oxygen, and sulfur. Examples of monocyclic (and monovalent) aryl groups include phenyl, pyrrolyl, furanyl, thiopheneyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Aryl and arylene groups also include bicyclic groups, tricyclic groups, etc., including fused 5- and 6-membered rings described above. Examples of multicyclic (and monovalent) aryl groups include naphthyl, biphenyl, anthracenyl, pyrenyl, carbazolyl, benzofuranyl, benzothiopheneyl, indolyl, benzoxazolyl, benzodioxazolyl, benzimidazolyl, indazolyl, benzotriazolyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolizinyl, imidazo[1,2-a]pyridinyl, imidazo[1,5-a]pyridinyl, pyrazolo[1,5-a]pyridinyl, pyrrolo[1,2-b]pyridinyl, and imidazo[1,2-c]pyridinyl. Other examples include quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, and pyrimido[4,5-d]pyrimidinyl.

The aryl and arylene groups may be attached to a parent group or to a substrate at any ring atom, unless such attachment would violate valence requirements. Likewise, aryl and arylene groups may include one or more non-hydrogen substituents unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

As used herein “heterocycle” and “heterocyclyl” refer to saturated, partially unsaturated, or unsaturated monocyclic or bicyclic rings having from 3 to 7 or from 7 to 11 ring members, respectively. These groups have ring members made up of carbon atoms and from 1 to 4 heteroatoms that are each independently selected from nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino. Examples of heterocycles include the aryl groups listed above which contain at least one heteroatom. Other examples include oxiranyl, thiaranyl, aziridinyl, oxetanyl, thiatanyl, azetidinyl, tetrahydrothiopheneyl, tetrahydropyran, tetrahydrothiopyran, 1,4-dioxanyl, 1,4-oxathianyl, 1,4-dithianyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 5,6-dihydro-2H-pyranyl, 2H-pyranyl, 1,2,3,4-tetrahydropyridinyl, 1,2,5,6-tetrahydropyridinyl, acridinyl, azocinyl, benzothiofuranyl, benzothiazolyl, benzotetrazolyl, benzoisoxazolyl, benzoisothiazolyl, benzoimidazolinyl, 4aH-carbazolyl, carbolinyl, chromanyl, chromenyl, decahydroquinolinyl, 2H,6H-1,5,2-dithiazinyl, dihydrofuro[2,3-b]tetrahydrofuranyl, furazanyl, imidazolidinyl, imidazolinyl, indolenyl, indolinyl, 3H-indolyl, isobenzofuranyl, isochromanyl, isoindazolyl, isoindolinyl, morpholinyl, octahydroisoquinolinyl, oxazolidinyl, oxazolidinyl, pyrimidinyl, phenanthridinyl, phenanthrolinyl, phenazinyl, phenothiazinyl, phenoxathiinyl, phenoxazinyl, piperazinyl, piperidinyl, pteridinyl, purinyl, pyranyl, pyrazolidinyl, pyrazolinyl, pyridooxazole, pyridoimidazole, pyridothiazole, pyridyl, pyrrolidinyl, pyrrolinyl, 2H-pyrrolyl, 4H-quinolizinyl, quinuclidinyl, tetrahydrofuranyl, tetrahydroisoquinolinyl, tetrahydroquinolinyl, 6H-1,2,5-thiadiazinyl, thianthrenyl, thienothiazolyl, thienooxazolyl, thienoimidazolyl, triazinyl, 1,2,4-triazolyl, 1,2,5-triazolyl, and xanthenyl.

The term “heteroaryl” as used herein is intended to denote 3-14 membered monocyclic or polycyclic ring systems having at least one aromatic ring that contains at least 1, and up to 4, ring heteroatoms independently selected from N, O and S. Heteroaryl groups can contain one or more non-aromatic rings fused to (i.e., sharing a bound in common with) the monocyclic or polycyclic heteroatom-containing ring described above, provided that the group is attached to the remainder of the molecule through the aromatic portion thereof. Thus, the term “heteroaryl” includes groups such as 5,6,7,8-tetrahydroquinolin-2-yl groups. Further examples of heteroaryl groups include furyl, thienyl, pyridyl, pyrrolyl, oxazolyl, thiazolyl, isothiazolyl, imidazolyl, pyrazolyl, isoxazolyl, triazolyl, oxadiazolyl, pyrimidinyl, pyrazinyl, indolyl, benzimidazolyl, benzothiophenyl, quinolinyl, isoquinolinyl, quinoxalinyl, and benzothiazolyl.

As used herein, “aryloxy” and “heteroaryloxy” refer, respectively, to aryl-O— and heteroaryl-O—, where aryl and heteroaryl are defined above. Examples include phenoxy, imidazol-2-yloxy, and the like.

The term “alkoxy” as used herein refers to a group of formula —O-alkyl. Examples of alkoxy groups include but are not limited to methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy, octoxy, prop-2-oxy, but-2-oxy and methylprop-2-oxy.

The terms “halo” or “halogen” refers to Cl, Br, F, and I.

The term “haloalkyl” is intended to include both branched and straight-chain saturated aliphatic hydrocarbon groups having the specified number of carbon atoms, substituted with 1 or more halogen atom. Haloalkyl groups include perhaloalkyl groups, wherein all hydrogens of an alkyl group have been replaced with halogens (e.g., —CF₃, —CF₂CF₃). The halogens can be the same (e.g., CHF₂, —CF₃) or different (e.g., CF₂Cl). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

Table A lists abbreviations used throughout the specification.

TABLE A
List of Abbreviations

| Abbreviation | Description |
|---|---|
| ACN | Acetonitrile |
| DMSO | Dimethylsulfoxide |
| EDCI | 1-ethyl-3-(3- |
| | dimethylaminopropyl)carbodiimide |
| ESI | electrospray ionization |
| EtOAc | ethyl acetate |
| HEK | human embryonic kidney |
| HEPES | 4-(2-hydroxyethyl)piperazine-1- |
| | ethanesulfonic acid |
| HOBT | Hydroxybenzotriazole |
| HPLC | high performance liquid |
| | chromatography |
| HRMS | high resolution mass spectrometry |
| IC₅₀ | concentration at 50% inhibition |
| Ki | inhibition constant |
| LCMS | liquid chromatography mass |
| | spectrometry |
| MeOH | methyl alcohol |
| MPEP | 2-methyl-6-(phenylethynyl)pyridine |
| Ms | mass spectrometry |
| NMR, s, d, t, q, m, | nuclear magnetic resonance, singlet, |
| | doublet, triplet, quartet, multiplet, |
| br | broad |
| PEI | polyethyleneimine |
| PyBOP | benzotriazol-1-yl- |
| | oxytripyrrolidinophosphonium |
| | hexafluorophosphate |
| TEA | triethylamine |
| TFA | trifluoroacetic acid |
| THF | tetrahydrofuran |
| Tris buffer | 2-amino-2-hydroxymethyl-propane- |
| | 1,3-diol buffer |

Methods of treating the diseases and syndromes listed herein are understood to involve administering to an individual in need of such treatment a therapeutically effective amount of a compound of the invention, or a salt, a hydrate or a solvate thereof, or a composition comprising one or more of the same. Accordingly, methods are provided in accordance with the invention for treating disorders involving the mGluR5 receptor, such as anxiety, pain, addiction, and depression diseases and/or disorders, including those specifically listed above, comprising the administration to a patient in need thereof a compound of the invention, or a pharmaceutically acceptable salt, hydrate or solvate thereof. Such methods comprise administering to the patient in need of such treatment a pharmaceutically or therapeutically effective amount of a compound of this invention. In the instances of combination therapies described herein, it will be understood the administration further includes a pharmaceutically or therapeutically effective amount of the second pharmaceutical agent in question. The second or additional pharmacological agents described herein may be administered in the doses and regimens known in the art.

As used herein, the phrase “therapeutically effective amount” refers to the amount of active compound or pharmaceutical agent that is effective to treat the condition of interest—i.e., the amount of active compound or pharmaceutical agent that is effective to elicit a biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds (including the salts) and the compositions of the present invention may be given at an oral dose of from about 0.1 mg/day to about 1,000 mg/day. Preferably, administration will be from about 10 mg/day to about 600 mg/day, more preferably from about 50 mg/day to about 600 mg/day. The dosing regimen can be adjusted to provide the optimal therapeutic response, and the projected daily dosages are expected to vary with route of administration. Several divided doses can be delivered daily or a single daily dosage can be delivered. The dose can be proportionally reduced or increased as indicated by the exigencies of the therapeutic situation.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

Therapeutic doses of compounds or compositions of the invention can be administered in any manner useful in directing the active compounds herein to the recipient's bloodstream. For example, compounds and compositions of the invention can be delivered by a route such as oral, via implants, dermal, transdermal, intrabronchial, intranasal, parental (including intravenous, intraperitoneal, intraarticularly and subcutaneous injections), intraperitoneal, sublingual, intracranial, epidural, intratracheal, vaginal, rectal, topical, ocular (via eye drops) or by sustained release. Optionally, one or more of the compounds of Formula I can be mixed with other active agents.

When the compound is delivered orally, it can be sub-divided in a dose containing appropriate quantities of the active ingredient. The unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form. The powders and tablets can contain up to 99% of the active ingredient.

The compounds of Formula I can be combined with one or more pharmaceutically acceptable carriers or excipients including, without limitation, solid and liquid carriers, which are compatible with the compounds of Formula I. Oral formulations containing the active compounds (including the salts, hydrates and solvates thereof) and the compositions of the present invention may comprise any conventionally used oral forms, including tablets, capsules, buccal forms, troches, lozenges and oral liquids, suspensions or solutions. Such carriers can include adjuvants, syrups, elixirs, diluents, binders, lubricants, surfactants, granulating agents, disintegrating agents, emollients, solubilizers, suspending agents, fillers, glidants, compression aids, encapsulating materials, emulsifiers, buffers, preservatives, thickening agents, colors, viscosity regulators, stabilizers, osmoregulators, and combinations thereof. Optionally, one or more of the compounds of Formula I can be mixed with other active agents.

Adjuvants can include, without limitation, flavoring agents, sweeteners, coloring agents, preservatives, and supplemental antioxidants, which can include vitamin E, ascorbic acid, butylated hydroxytoluene (BHT) and butylated hydroxyanisole (NHA).

Elixirs and syrups can be prepared from acceptable sweeteners such as sugar, saccharine or a biological sweetener, a flavoring agent, and/or solvent.

Capsules and tablets may contain mixtures of the active compound(s) with inert fillers, diluents, binders, lubricants, granulating agents, disintegrating agents, emollients, surface modifying agents (including surfactants), suspending or stabilizing agents, and the like. Nonlimiting examples of diluents and fillers include materials in which the compound can be dispersed, dissolved, or incorporated, such as water, lower monovalent alcohols, polyhydric alcohols, and low molecular weight glycols and polyols, including, for example, propylene glycol, glycerol, butylenes glycol, 1,2,4-butanetriol, sorbitol esters, 1,2,6-hexanetriol, ethanol, isopropanol, butanediol, ethyl oleate, isopropyl myristate, ether propanol, ethoxylated ethers, propoxylated ethers, oils such as corn, peanut, fractionated coconut, arachis, sesame oils, dimethylsulfoxide (DMSO), dimethylformamide (DMF), waxes, dextrin, and combinations thereof. Examples of binders include, without limitation, cellulose, methylcellulose, hydroxymethylcellulose, polypropylpyrrolidone, polyvinylpyrrolidone, polyvinylpyrrolidine, gelatin, gum Arabic, polyethylene glycol, starch, sugars such as, for example, sucrose kaoline, cellulose kaolin, and lactose. Nonlimiting examples of surface modifying agents include, but are not limited to, poloxamer 188, benzalkonium chloride, calcium stearate, cetostearl alcohol, sorbitan esters, colloidal, silicon dioxide, phosphates, sodium dodecylsulfate, magnesium aluminum silicate, lauryl sulfates, and triethanolamine. Examples of lubricants include, without limitation, magnesium stearate, light anhydrous silicic acid, talc and sodium lauryl sulfate. Examples of granulating agents include, without limitation, silicon dioxide, microcrystalline cellulose, starch, calcium carbonate, pectin, crospovidone, and polyplasdone. Examples of disintegrating agents include, without limitation, pharmaceutically acceptable starches (e.g. corn, potato or tapioca starch), carboxymethylcellulose, hydroxypropylstarch, substituted hydroxypropylcellulose, sodium bicarbonate, calcium phosphate, and calcium citrate. Examples of emollients include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents as described above.

Oral formulations herein may utilize standard delay or time-release formulations to alter the absorption of the active compound(s). The oral formulation may also consist of administering the active ingredient in water or a fruit juice, containing appropriate solubilizers or emulsifiers as needed.

In some cases it may be desirable to administer the compounds (including the salts) and the compositions of the present invention directly to the airways in the form of an aerosol.

The compounds (including salts, hydrates and solvates) and the compositions of the present invention may also be administered parenterally or intraperitoneally. Solutions or suspensions of these active compounds (including the salts) and the compositions of the present invention can be prepared in water optionally mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

In some embodiments, sustained delivery devices can be used, in order to avoid the necessity to take medications on a daily basis. The term “sustained delivery” is used herein to refer to delaying the release of an active agent, i.e., a compound of Formula I, until after placement in a delivery environment, followed by a sustained release of the agent at a later time. A number of sustained delivery devices are known in the art and include, for example, hydrogels (U.S. Pat. Nos. 5,266,325; 4,959,217; 5,292,515), osmotic pumps (U.S. Pat. Nos. 4,295,987 and 5,273,752 and European Pat. No. 314,206, among others; hydrophobic membrane materials, such as ethylenemethacrylate (EMA) and ethylenevinylacetate (EVA); bioresorbable polymer systems (International Patent Publication No. WO 98/44964 and U.S. Pat. Nos. 5,756,127 and 5,854,388); and other bioresorbable implant devises composed of, for example, polyesters, polyanhydrides, or lactic acid/glycolic acid copolymers (U.S. Pat. No. 5,817,343). For use in such sustained delivery devices, the compounds of the invention can be formulated as described herein.

Suppository formulations may be made from traditional materials, including cocoa butter, with or without the addition of waxes to alter the suppository's melting point, and glycerin. Water-soluble suppository bases, such as polyethylene glycols of various molecular weights, may also be used.

Additional numerous various excipients, dosage forms, dispersing agents and the like that are suitable for use in connection with the salt forms of the invention are known in the art and described in, for example, Remington's Pharmaceutical Sciences, 17th ed., Mack Publishing Company, Easton, Pa., 1985, which is incorporated herein by reference in its entirety.

The compounds of Formula I have utility for the repression and/or treatment of disorders involving the mGluR5 receptor, such as anxiety and depression disorders. Examples of disorders or conditions which can be treated by the compounds, compositions and methods of this invention include depression, anxiety (including panic disorders, social anxiety, obsessive compulsive disorders, generalized anxiety disorders, phobias), post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia and obesity.

The compounds of Formula I have utility for the repression and/or treatment of disorders involving the mGluR5 receptor, such as pain and addiction disorders. Examples of disorders or conditions which can be treated by the compounds, compositions and methods of this invention include acute pain, chronic pain, neuropathic pain, nociceptive pain, and inflammatory pain. Addiction disorders can include, for example, drug addiction; alcohol addiction, gambling addiction, and sexual addiction.

### Preparation of Compounds of the Invention

### General Preparative Schemes

Compounds of the invention can be prepared using the four general schemes outlined below, together with synthetic methods known in the synthetic organic arts or variations of these methods by one skilled in the art. Some of the schemes and examples may omit details of common reactions, including oxidations, reductions, and so on, separation techniques, and analytical procedures, which are known to persons of ordinary skill in the art of organic chemistry. The details of such reactions and techniques can be found in a number of treatises, including Richard Larock, Comprehensive Organic Transformations (1999), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a diacid, etc.). In addition, in some instances, reaction intermediates may be used in subsequent steps without isolation or purification (i.e., in situ).

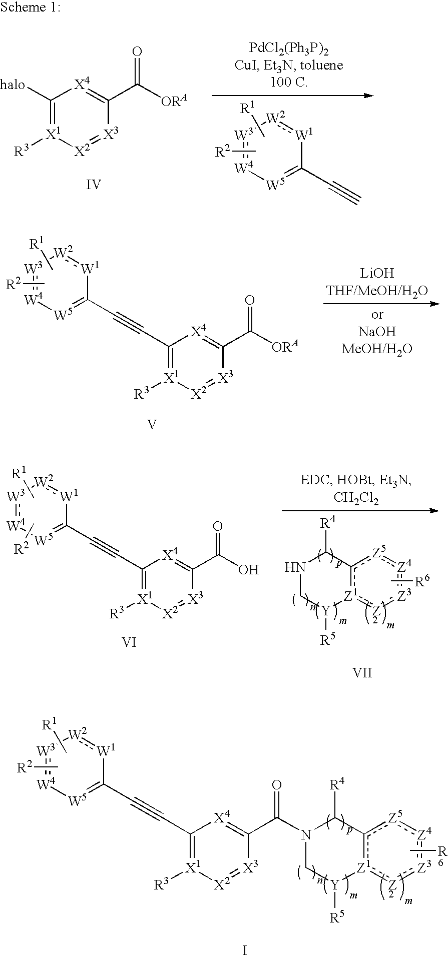

Target molecules (I) can be prepared according to Scheme 1. Sonagashira coupling of substituted or unsubstituted 3-halobenzoates with appropriately substituted terminal alkynes affords the desired acetylenes (V). Basic hydrolysis of the ester moiety in V with either LiOH or NaOH under standard conditions provides the carboxylic acids (VI). The acids can be coupled with appropriately substituted cyclic amines (VII), which are either commercially available or can be prepared by methods outlined in the literature, to provide the target amides (I). The coupling can be achieved using standard EDCl peptide coupling conditions or other related methods for amide formation.

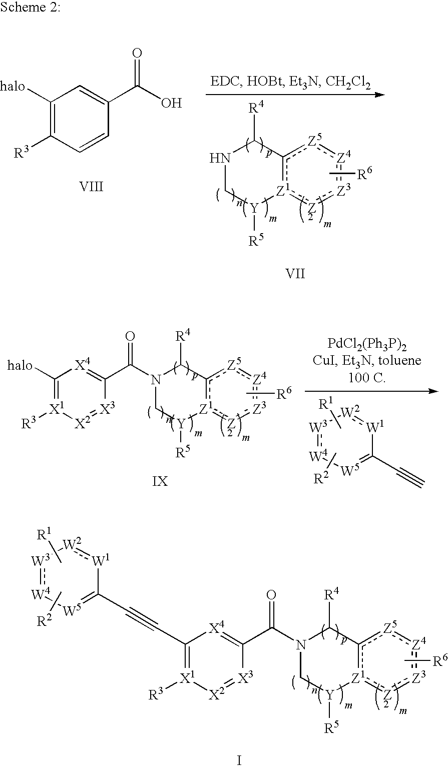

Alternatively, target molecules (I) may also be prepared according to Scheme 2, whereby the amide is prepared prior to the introduction of the acetylene moiety via a Sonagashira reaction. Readily available 3-halobenzoic acids can be coupled with appropriately substituted cyclic amines (VII) using standard EDCl peptide coupling conditions or other related methods to provide amides (IX). Sonagashira coupling of IX with appropriately substituted terminal alkynes affords the target the target molecules (I).

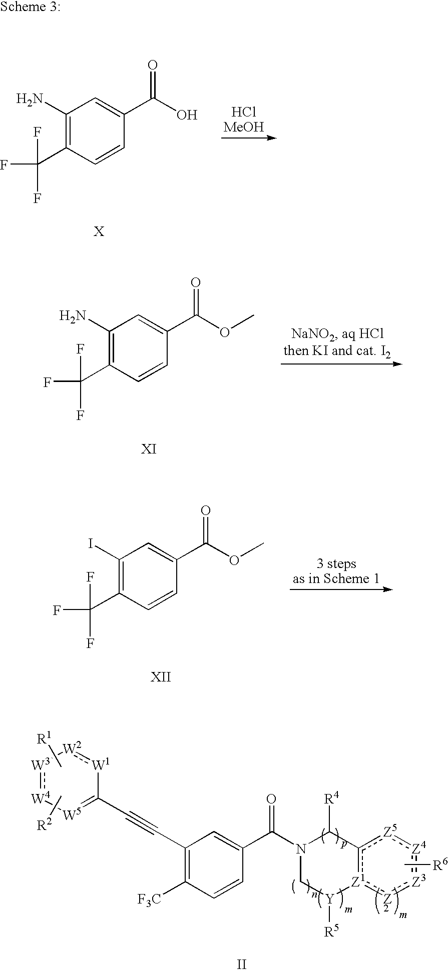

Target molecules of type II can be prepared according to Scheme 3. Commercially available 3-amino-4-(trifluoromethyl)benzoic acid (X) can be converted to methyl ester XI upon treatment with HCl in MeOH. Diazotization and conversion to aryl iodide XII can be accomplished by sequential treatment of XI with NaNO₂ in aqueous HCl followed by treatment with KI and catalytic I₂. Conversion of XII to the target molecules (II) can be achieved by either of the sequences outlined in Schemes 1 or 2.

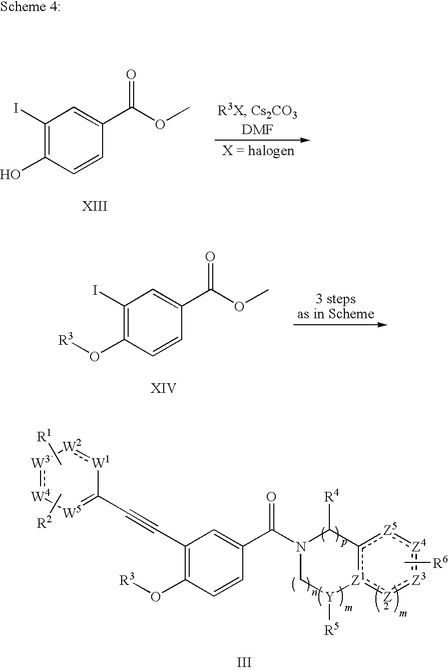

Target molecules of type III can be prepared according to Scheme 4. Commercially available methyl 4-hydroxy-3-iodobenzoate (XIII) can be alkylated upon treatment with an appropriately substituted alkyl halide and Cs₂CO₃ to provide intermediates XIV. The aryl iodides (XIV) can be converted to the target molecules (III) by either of the sequences outlined in Schemes 1 or 2.

Accordingly, compounds of Formula I

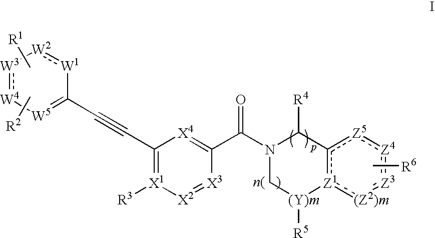

or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

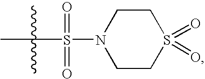

the C₁-C₆alkyl, C₃₋₆cycloalkyl, aryl, heteroaryl, aryloxy, heteroaryloxy, C₁-C₆alkylNR⁷R⁸ and

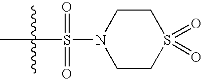

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S,
are made by reacting a compound of Formula VI:

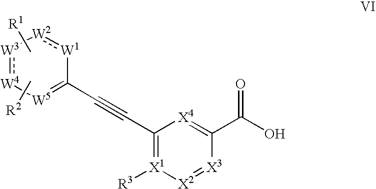

with an cyclic amine of Formula VII:

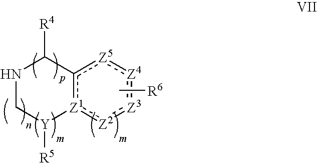

for a time and under conditions effective to form the compound of Formula I.

Compounds of Formula I:

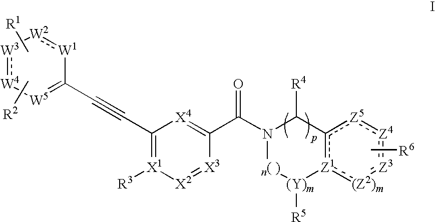

or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

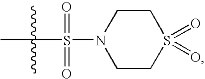

the C₁-C₆alkyl, C₃-C₆cycloalkyl, aryl, heteroaryl, aryloxy, heteroaryloxy, C₁-C₆alkylNR⁷R⁸ and

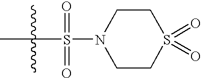

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S,
can also be made by reacting a compound of Formula IX:

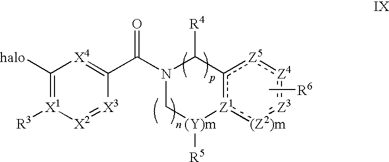

where halo is halogen, with an acetylene of Formula:

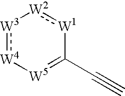

in the presence of a catalyst such as a palladium triphenylphosphine-containing catalyst for a time and under conditions effective to form the compound of Formula I. In this process, the palladium triphenylphosphine-containing catalyst can be Pd Cl₂(Ph₃P)₂.

Compounds of Formula II:

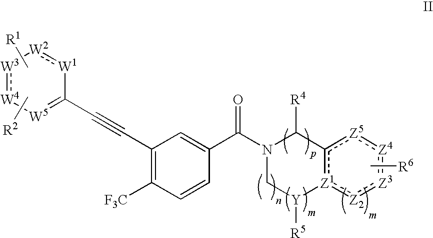

wherein:
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

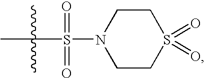

the C₁-C₆alkyl, C₃-C₆cycloalkyl, aryl, heteroaryl, aryloxy, heteroaryloxy, C₁-C₆alkylNR⁷R⁸ and

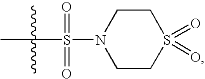

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S,
can be made by reacting a compound of Formula XII:

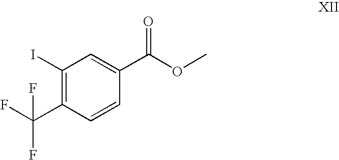

with a compound of Formula:

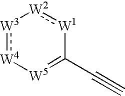

where W¹-W⁵ are each independently selected from C, N, S, and O; [symbol]
is independently a single bond or a double bond; and
R¹ and R² are each independently selected from H, hydroxyl, halogen, cyano, C₁-C₆alkyl,
halo-C₁-C₆alkyl, and —O(C₁-C₆alkyl) in the presence of a palladium triphenylphosphine-containing catalyst for a time and under conditions effective to form a compound of Formula Va:

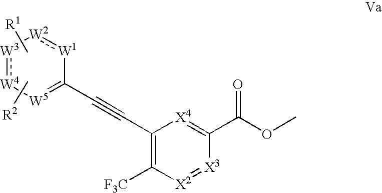

where W¹-W⁵ and X²-X⁴ are each independently selected from C, N, S, and O; [symbol]
is independently a single bond or a double bond;
R¹ and R² are each independently selected from H, hydroxyl, halogen, cyano, C₁-C₆alkyl, halo-C₁-C₆alkyl, and —O(C₁-C₆alkyl);
reacting the compound of Formula Va for a time and under conditions effective to form a compound of Formula VIa:

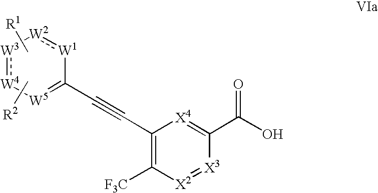

reacting the compound of Formula VIa with a compound of Formula VIIa:

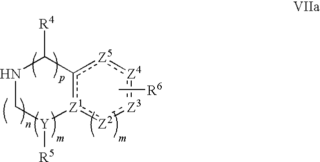

where Z¹-Z⁵ are each independently selected from C, N, S, and O; [symbol]
is independently a single bond or a double bond;
n is 1 or 2;
m is 0 or 1;
p is 1 or 2;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

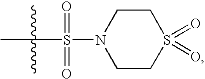

the C₁-C₆alkyl, C₃-C₆cycloalkyl, aryl, heteroaryl, aryloxy, heteroaryloxy, C₁-C₆alkylNR⁷R⁸ and

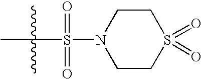

groups are optionally independently substituted with from 1 to 4-C₁-C₆alkyl, halo-C₁-C₆alkyl, —OH, or —NH₂; or R⁴ and R⁵ and the atoms to which they are attached form a ring;
and
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino to form the compound of Formula II.

In this process, the palladium triphenylphosphine-containing catalyst can be Pd Cl₂(Ph₃P)₂.

Compounds of Formula III:

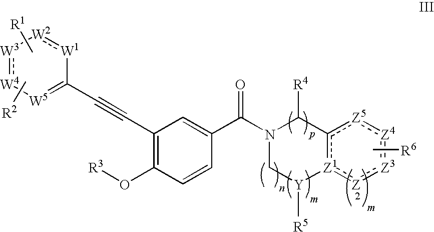

wherein:
W¹-W⁵, Y and Z¹-Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, C₁-C₆alkyl and halo-C₁-C₆alkyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

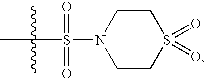

the C₁-C₆alkyl, C₃-C₆cycloalkyl, aryl, heteroaryl, aryloxy, heteroaryloxy, C₁-C₆alkylNR⁷R⁸ and

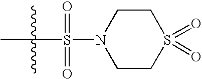

groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S,
can be made by reacting a compound of Formula XIV:

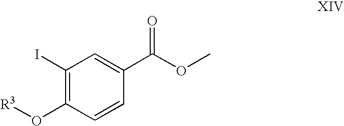

R³ is selected from H, C₁-C₆alkyl, and halo-C₁-C₆alkyl;
with a compound of Formula:

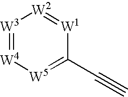

where W¹-W⁵ are each independently selected from C, N, S, and O; [symbol]
is independently a single bond or a double bond; and
R¹ and R² are each independently selected from H, hydroxyl, halogen, cyano, C₁-C₆alkyl, halo-C₁-C₆alkyl, and —O(C₁-C₆alkyl) in the presence of a palladium triphenylphosphine-containing catalyst for a time and under conditions effective to form a compound of Formula Vaa:

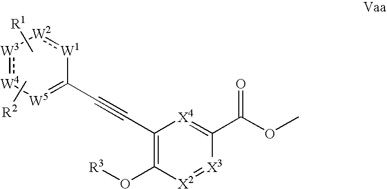

where W¹-W⁵ and X²-X⁴ are each independently selected from C, N, S, and O; [symbol]
is independently a single bond or a double bond;
R¹ and R² are each independently selected from H, hydroxyl, halogen, cyano, C₁-C₆alkyl, halo-C₁-C₆alkyl, and —O(C₁-C₆alkyl);
R³ is selected from H, C₁-C₆alkyl, and halo-C₁-C₆alkyl;
reacting the compound of Formula Vaa for a time and under conditions effective to form a compound of Formula VIaa:

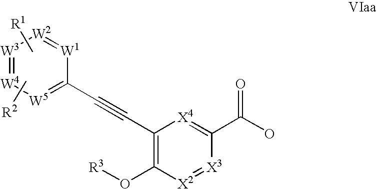

and reacting the compound of Formula VIaa with a compound of Formula VIIaa:

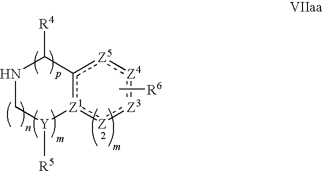

where Z¹-Z⁵ are each independently selected from C, N, S, and O; —
[symbol] is independently a single bond or a double bond;
   n is 1 or 2;
   m is 0 or 1;
   p is 1 or 2;
   R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and

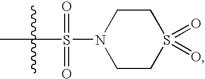

the C₁-C₆alkyl, C₃-C₆cycloalkyl, aryl, heteroaryl, aryloxy, heteroaryloxy, C₁-C₆alkylNR⁷R⁸ and

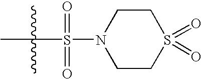

groups are optionally independently substituted with from 1 to 4-C₁-C₆alkyl, halo-C₁-C₆alkyl, —OH, or —NH₂; or R⁴ and R⁵ and the atoms to which they are attached form a ring; and
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino to form the compound of Formula III.

In this process, the palladium triphenylphosphine-containing catalyst can be Pd Cl₂(Ph₃P)₂.

### Analytical Methods

The following methods were used for the characterization of compounds appearing in the Examples below.

### Standard LCMS Conditions for Compound Characterization:

### HPLC Conditions: Instrument—Agilent 1100

### Column: Thermo Aquasil C18, 50×2.1 mm, 5 μm

Mobile Phase A: 0.1% Formic Acid in water
B: 0.1% Formic Acid in ACN
   Flow Rate: 0.800 mL/min

### Column Temperature: 40° C.

### Injection Volume: 5 mL

UV: monitor 215, 230, 254, 280, and 300 nm
Purity is reported at 254 nm unless otherwise noted.

Gradient Table:

| Time (min) | % B |
|---|---|
| 0 | 0 |
| 2.5 | 100 |
| 4.0 | 100 |
| 4.1 | 0 |
| 5.5 | 0 |

MS Conditions: Instrument: Agilent MSD; Ionization Mode: API-ES; Gas Temperature: 350° C.; Drying Gas: 11.0 L/min.; Nebulizer Pressure: 55 psig; Polarity: 50% positive, 50% negative; VCap: 3000 V (positive), 2500 V (negative); Fragmentor: 80 (positive), 120 (negative); Mass Range: 100-1000 m/z; Threshold: 150; Step size: 0.15; Gain: 1; Peak width: 0.15 minutes.
Preparative reverse-phase HPLC (RP-HPLC): Compounds were in dissolved in 2 mL of 1:1 DMSO:MeCN, filtered through a 0.45 μm GMF, and purified on a Gilson HPLC, using a Phenomenex LUNA C₁₈ column: 60 mm×21.2 mm I.D., 5 um particle size: with ACN/H₂O (containing 0.2% TFA) gradient elution (95:5 H₂O:MeCN to 10:90 H₂O:MeCN; 8 minute run.

### Preparation of Exemplary Compounds

The following examples are provided to illustrate the production and activity of representative compounds of the present teachings and to illustrate their performance in a screening assay. One skilled in the art will appreciate that although specific reagents and conditions are outlined in the following examples, these reagents and conditions are not a limitation on the present teachings. In the following examples, chemical structures and names were produced using Chemdraw v 7.0.3. In any conflict between chemical nomenclature and structure, the structure should prevail.

### EXAMPLES

### Example 1

### 2-[4-Methoxy-3-(pyridin-2-ylethynyl)benzoyl]isoindoline

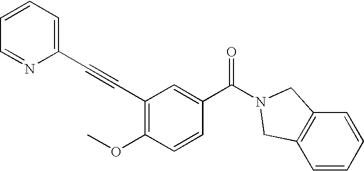

### Step 1: Methyl 4-methoxy-3-(pyridin-2-ylethynyl)benzoate

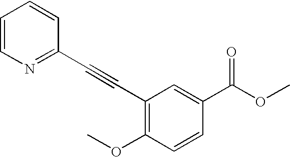

Methyl 3-iodo-4-methoxybenzoate (6.0 g, 20.4 mmol), 2-ethynylpyridine (3.14 mL, 31.1 mmol), and triethylamine (6.2 mL, 44.7 mmol) were dissolved in 100 mL of toluene and purged with nitrogen. Then, CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (20:1 CH₂Cl₂/EtOAc) to yield 5.3 g (96%) of product as a brown solid.

### Step 2: 4-Methoxy-3-(pyridin-2-ylethynyl)benzoic acid

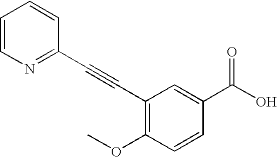

The methyl ester (5.3 g, 20 mmol) was dissolved in a mixture of THF (150 mL), MeOH (20 mL), and H₂O (40 mL) and treated with lithium hydroxide monohydrate (1.68 g, 40 mmol). The reaction was stirred at room temperature overnight and then concentrated at reduced pressure to an approximate volume of 40 mL. The remaining solution was diluted with an additional 50 mL of H₂O, washed with Et₂O (×2), and acidified to pH 4.0. The resulting precipitate was collected by suction filtration. The filtrate was saturated with solid NaCl and extracted with EtOAc (2×100 mL). The organic extracts were concentrated to yield a solid residue that was added to the collected precipitate and the combined solids are dried in a vacuum oven at 50° C. for 3 hours to yield 4.65 g (93%) of the carboxylic acid as a tan solid. No additional purification of the carboxylic acid was required.

### Step 3: 2-[4-Methoxy-3-(pyridin-2-ylethynyl)benzoyl]isoindoline

A 50 mL, round-bottom flask equipped with a magnetic stirbar was charged with 4-methoxy-3-(pyridin-2-ylethynyl)benzoic acid (400 mg, 1.58 mmol), HOBT (387 mg, 2.53 mmol) and EDC (454 mg, 2.37 mmol). The reagents were dissolved in 12 mL CH₂Cl₂ and the solution was allowed to stir for 15 min. Subsequently, isoindoline (0.233 mL, 2.05 mmol) and TEA (0.657 mL, 4.74 mmol) were added to the reaction vessel and the solution was allowed to stir for 5 h. Upon completion, the solvent was removed under reduced pressure and the residue was purified via flash chromatography on silica gel (20:1 CH₂Cl₂/MeOH) to afford 415 mg (74%) of the title compound as a white solid. ¹H NMR (400 MHz, CDCl₃): consistent; MS (ES⁺) m/z 355.1 (M+H).

A procedure similar to that of Example 1, substituting the required cyclic secondary amine for isoindoline provides Examples 2-59. The compounds and their analytical data are shown in Table 1.

TABLE 1
Compounds Prepared According to the Procedure of Example 1

| Example | Compound Name | MS |
|---|---|---|
| 2 | 2-[4-methoxy-3-(pyridin-2-ylethynyl)benzoyl]- | (ESI) m/z 369.2 (M + H) |
| | 1,2,3,4-tetrahydroisoquinoline | |
| 3 | 5-fluoro-2-[4-methoxy-3-(pyridin-2- | (ESI) m/z 387.1 (M + H) |
| | ylethynyl)benzoyl]-1,2,3,4-tetrahydroisoquinoline | |
| 4 | 2-[4-methoxy-3-(pyridin-2-ylethynyl)benzoyl]-5- | (ESI) m/z 437.1 (M + H) |
| | (trifluoromethyl)-1,2,3,4-tetrahydroisoquinoline | |
| 5 | 2-[4-methoxy-3-(pyridin-2-ylethynyl)benzoyl]-8- | (ESI) m/z 437.1 (M + H) |
| | (trifluoromethyl)-1,2,3,4-tetrahydroisoquinoline | |
| 6 | 5,8-difluoro-2- | (ESI) m/z 405.1 (M + H) |
| | [4-methoxy-3-(pyridin-2-ylethynyl)benzoyl]- | |
| | 1,2,3,4-tetrahydroisoquinoline | |
| 7 | 5,7-dichloro-2-[4-methoxy-3-(pyridin-2- | (ESI) m/z 437.0 (M + H) |
| | ylethynyl)benzoyl]-1,2,3,4-tetrahydroisoquinoline | |
| 8 | 7-chloro-2-[4-methoxy-3-(pyridin-2- | (ESI) m/z 403.1 (M + H) |
| | ylethynyl)benzoyl]- | |
| | 1,2,3,4-tetrahydroisoquinoline | |
| 9 | 7-fluoro-2-[4-methoxy-3-(pyridin-2- | (ESI) m/z 487.1 (M + H) |
| | ylethynyl)benzoyl]- | |
| | 1,2,3,4-tetrahydroisoquinoline | |
| 10 | 5-chloro-2-[4-methoxy-3-(pyridin-2- | (ESI) m/z 403.1 (M + H) |
| | ylethynyl)benzoyl]- | |
| | 1,2,3,4-tetrahydroisoquinoline | |
| 11 | 2-[4-methoxy-3-(pyridin-2-ylethynyl)benzoyl]- | (ESI) m/z 394.1 (M + H) |
| | 1,2,3,4-tetrahydroisoquinoline-7-carbonitrile | |
| 12 | 2-[4-methoxy-3-(pyridin-2-ylethynyl)benzoyl]- | (ESI) m/z 408.1 (M + H) |
| | 2,3,4,5-tetrahydro-1H-pyrido[4,3-b]indole | |
| 13 | 5-[4-methoxy-3-(pyridin-2-ylethynyl)benzoyl]- | (ESI) m/z 375.0 (M + H) |
| | 4,5,6,7-tetrahydrothieno[3,2-c]pyridine | |
| 14 | 3-bromo-6-[4-methoxy-3-(pyridin-2- | (ESI) m/z 448.0 (M + H) |
| | ylethynyl)benzoyl]- | |
| | 5,6,7,8-tetrahydro-1,6-naphthyridine | |
| 15 | 6-[4-methoxy-3-(pyridin-2-ylethynyl)benzoyl]- | (ESI) m/z 385.1 (M − H) |
| | 5,6,7,8-tetrahydropyrido[4,3-d]pyrimidin-4(3H)-one | |
| 16 | 2-[4-methoxy-3-(pyridin-2-ylethynyl)benzoyl]-7- | (ESI) m/z 388.1 (M + H) |
| | (trifluoromethyl)-1,2,3,4-tetrahydroisoquinoline | |
| 17 | 1-{2-[4-methoxy-3-(pyridin-2-ylethynyl)benzoyl]- | (ESI) m/z 427.2 (M + H) |
| | 2,3- | |
| | dihydro-1H-isoindol-1-yl}-2-methylpropan-2-ol | |
| 18 | 9-[4-methoxy-3-(pyridin-2-ylethynyl)benzoyl]- | (ESI) m/z 381.2 (M + H) |
| | 1,2,3,4-tetrahydro-1,4-epiminonaphthalene | |
| 19 | 5,8-dimethoxy-2-[4-methoxy-3-(pyridin-2- | (ESI) m/z 429.2 (M + H) |
| | ylethynyl)benzoyl]-1,2,3,4-tetrahydroisoquinoline | |
| 20 | 6,7-dimethoxy-2-[4-methoxy-3-(pyridin-2- | (ESI) m/z 429.2 (M + H) |
| | ylethynyl)benzoyl]-1,2,3,4-tetrahydroisoquinoline | |
| 21 | 8-methoxy-2-[4-methoxy-3-(pyridin-2- | (ESI) m/z 399.2 (M + H) |
| | ylethynyl)benzoyl]-1,2,3,4-tetrahydroisoquinoline | |
| 22 | 2-[4-methoxy-3-(pyridin-2-ylethynyl)benzoyl]-1- | (ESI) m/z 451.2 (M + H) |
| | (2-thienyl)-1,2,3,4-tetrahydroisoquinoline | |
| 23 | 2-[4-methoxy-3-(pyridin-2-ylethynyl)benzoyl]-8-[1- | (ESI) m/z 530.2 (M + H) |
| | (methylsulfonyl)piperidin-4-yl]-1,2,3,4- | |
| | tetrahydroisoquinoline | |
| 24 | 2-[4-methoxy-3-(pyridin-2-ylethynyl)benzoyl]-4-[4- | (ESI) m/z 529.2 (M + H) |
| | (trifluoromethyl)phenoxy]-1,2,3,4- | |
| | tetrahydroisoquinoline | |
| 25 | 4-[(1,1-dioxidothiomorpholin-4-yl)sulfonyl]-2-[4- | (ESI) m/z 566.1 (M + H) |
| | methoxy-3-(pyridin-2-ylethynyl)benzoyl]-1,2,3,4- | |
| | tetrahydroisoquinoline | |
| 26 | 2-[4-methoxy-3-(pyridin-2-ylethynyl)benzoyl]-1- | (ESI) m/z 445.2 (M + H) |
| | phenyl-1,2,3,4-tetrahydroisoquinoline | |
| 27 | 1-(2-furyl)-2-[4-methoxy-3-(pyridin-2- | (ESI) m/z 435.2 (M + H) |
| | ylethynyl)benzoyl]-1,2,3,4-tetrahydroisoquinoline | |
| 28 | (1S)-2-[4-methoxy-3-(pyridin-2-ylethynyl)benzoyl]- | (ESI) m/z 439.2 (M + H) |
| | 1- | |
| | [(2S)-tetrahydrofuran-2-yl]-1,2,3,4- | |
| | tetrahydroisoquinoline | |
| 29 | N-({2-[4-methoxy-3-(pyridin-2-ylethynyl)benzoyl]- | (ESI) m/z 474.2 (M + H) |
| | 1,2,3,4-tetrahydroisoquinolin-4-yl}methyl)aniline | |
| 30 | 1-benzyl-2-[4-methoxy-3-(pyridin-2- | (ESI) m/z 459.2 (M + H) |
| | ylethynyl)benzoyl]- | |
| | 1,2,3,4-tetrahydroisoquinoline | |
| 31 | 6-methoxy-2-[4-methoxy-3-(pyridin-2- | (ESI) m/z 399.2 (M + H) |
| | ylethynyl)benzoyl]-1,2,3,4-tetrahydroisoquinoline | |
| 32 | 2-[4-methoxy-3-(pyridin-2-ylethynyl)benzoyl]-5- | (ESI) m/z 369.2 (M + H) |
| | methylisoindoline | |
| 33 | 5-bromo-2-[4-methoxy-3-(pyridin-2- | (ESI) m/z 433.1 (M + H) |
| | ylethynyl)benzoyl]isoindoline | |
| 34 | 5,6-dichloro-2-[4-methoxy-3-(pyridin-2- | (ESI) m/z 423.1 (M + H) |
| | ylethynyl)benzoyl]isoindoline | |
| 35 | 2-[4-methoxy-3-(pyridin-2-ylethynyl)benzoyl]-5- | (ESI) m/z 400.1 (M + H) |
| | nitroisoindoline | |
| 36 | 4-fluoro-2-[4-methoxy-3-(pyridin-2- | (ESI) m/z 373.1 (M + H) |
| | ylethynyl)benzoyl]isoindoline | |
| 37 | 5-fluoro-2-[4-methoxy-3-(pyridin-2- | (ESI) m/z 373.1 (M + H) |
| | ylethynyl)benzoyl]isoindoline | |
| 38 | 4-chloro-2-[4-methoxy-3-(pyridin-2- | (ESI) m/z 389.1 (M + H) |
| | ylethynyl)benzoyl]isoindoline | |
| 39 | 6-[4-methoxy-3-(pyridin-2-ylethynyl)benzoyl]- | (ESI) m/z 370.2 (M + H) |
| | 5,6,7,8-tetrahydro-1,6-naphthyridine | |
| 40 | 6-[4-methoxy-3-(pyridin-2-ylethynyl)benzoyl]-3- | (ESI) m/z 384.2 (M + H) |
| | methyl-5,6,7,8-tetrahydro-1,6-naphthyridine | |
| 41 | 2-[4-methoxy-3-(pyridin-2-ylethynyl)benzoyl]-6- | (ESI) m/z 370.2 (M + H) |
| | methyl-2,3-dihydro-1H-pyrrolo[3,4-c]pyridine | |
| 42 | 2-[4-methoxy-3-(pyridin-2-ylethynyl)benzoyl]-6,7- | (ESI) m/z 384.2 (M + H) |
| | dimethyl-2,3-dihydro-1H-pyrrolo[3,4-c]pyridine | |
| 43 | 7-chloro-2-[4-methoxy-3-(pyridin-2- | (ESI) m/z 404.1 (M + H) |
| | ylethynyl)benzoyl]-6-methyl-2,3-dihydro-1H- | |
| | pyrrolo[3,4-c]pyridine | |
| 44 | 2-[4-methoxy-3-(pyridin-2-ylethynyl)benzoyl]-6- | (ESI) m/z 385.2 (M + H) |
| | methyl-2,3-dihydro-1H-pyrrolo[3,4-c]pyridin-7- | |
| | amine | |
| 45 | 7-methoxy-2-[4-methoxy-3-(pyridin-2- | (ESI) m/z 400.2 (M + H) |
| | ylethynyl)benzoyl]-6-methyl-2,3-dihydro-1H- | |
| | pyrrolo[3,4-c]pyridine | |
| 46 | 2-[4-methoxy-3-(pyridin-2-ylethynyl)benzoyl]-2,3- | (ESI) m/z 356.1 (M + H) |
| | dihydro-1H-pyrrolo[3,4-c]pyridine | |
| 47 | 5-{[4-methoxy-3-(pyridin-2- | (ESI) m/z 374.2 (M + H) |
| | ylethynyl)phenyl]carbonyl}-2-methyl-4,5,6,7- | |
| | tetrahydro[1,3]oxazolo[4,5-c]pyridine | |
| 48 | 5-{[4-methoxy-3-(pyridin-2- | (ESI) m/z 390.1 (M + H) |
| | ylethynyl)phenyl]carbonyl}-2-methyl-4,5,6,7- | |
| | tetrahydro[1,3]thiazolo[5,4-c]pyridine | |
| 49 | 7-{[4-methoxy-3-(pyridin-2- | (ESI) m/z 428.1 (M + H) |
| | ylethynyl)phenyl]carbonyl}-3- | |
| | (trifluoromethyl)-5,6,7,8- | |
| | tetrahydro[1,2,4]triazolo[4,3-a]pyrazine | |
| 50 | 7-{[4-methoxy-3-(pyridin-2- | (ESI) m/z 370.2 (M + H) |
| | ylethynyl)phenyl]carbonyl}-5,6,7,8-tetrahydro-1,7- | |
| | naphthyridine | |
| 51 | 3-chloro-6-{[4-methoxy-3-(pyridin-2- | (ESI) m/z 404.1 (M + H) |
| | ylethynyl)phenyl]carbonyl}-5,6,7,8-tetrahydro-1,6- | |
| | naphthyridine | |
| 52 | 6-{[4-methoxy-3-(pyridin-2- | (ESI) m/z 438.1 (M + H) |
| | ylethynyl)phenyl]carbonyl}-3- | |
| | (trifluoromethyl)-5,6,7,8-tetrahydro-1,6- | |
| | naphthyridine | |
| 53 | 5-{[4-methoxy-3-(pyridin-2- | (ESI) m/z 359.2 (M + H) |
| | ylethynyl)phenyl]carbonyl}-4,5,6,7-tetrahydro-1H- | |
| | pyrazolo[4,3-c]pyridine | |
| 54 | 6-{[4-methoxy-3-(pyridin-2- | (ESI) m/z 356.1 (M + H) |
| | ylethynyl)phenyl]carbonyl}-6,7-dihydro-5H- | |
| | pyrrolo[3,4-b]pyridine | |
| 55 | 4-{[4-methoxy-3-(pyridin-2- | (ESI) m/z 385.2 (M + H) |
| | ylethynyl)phenyl]carbonyl}-2,3,4,5-tetrahydro-1,4- | |
| | benzoxazepine | |
| 56 | 4-{[4-methoxy-3-(pyridin-2- | (ESI) m/z 384.2 (M + H) |
| | ylethynyl)phenyl]carbonyl}-2,3,4,5-tetrahydro-1H- | |
| | 1,4-benzodiazepine | |
| 57 | 3-{[4-methoxy-3-(pyridin-2- | (ESI) m/z 383.2 (M + H) |
| | ylethynyl)phenyl]carbonyl}-2,3,4,5-tetrahydro-1H- | |
| | 3-benzazepine | |
| 58 | 5-{[4-methoxy-3-(pyridin-2- | (ESI) m/z 359.2 (M + H) |
| | ylethynyl)phenyl]carbonyl}-4,5,6,7-tetrahydro-1H- | |
| | imidazo[4,5-c]pyridine | |
| 59 | 2-{[4-methoxy-3-(pyridin-2- | (ESI) m/z 383.2 (M + H) |
| | ylethynyl)phenyl]carbonyl}-2,3,4,5-tetrahydro-1H- | |
| | 2-benzazepine | |

### Example 60

### 2-[4-Chloro-3-(pyridin-2-ylethynyl)benzoyl]isoindoline

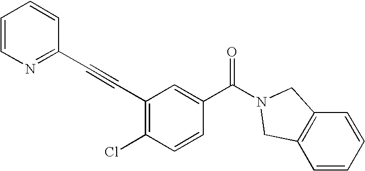

### Step 1: Methyl 4-chloro-3-(pyridin-2-ylethynyl)benzoate

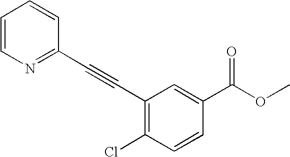

Methyl 3-bromo-4-chlorobenzoate (1.758 g, 7.089 mmol), 2-ethynyl pyridine (1.40 mL, 13.9 mmol), and triethylamine (2.20 mL, 15.8 mmol) were dissolved in 34 mL dry toluene. Nitrogen gas was bubbled through the mixture for 10 minutes, and then dichlorobis(triphenylphosphine)-palladium(II) (1.00 g, 1.42 mmol) and copper(I) iodide (0.268 g, 1.41 mmol) was added to the mixture. Nitrogen was bubbled through the mixture for another 5 minutes, and then the mixture was then heated to 100° C. for 6 hours. The mixture was cooled, and then filtered through a pad of Celite. The Celite pad was washed with ethyl acetate (2×) and then ˜5% methanol/methylene chloride 2×). The combined filtrate was evaporated and the residue was chromatographed on silica gel using a gradient elution of ethyl acetate in methylene chloride. Methyl 4-chloro-3-(pyridin-2-ylethynyl)benzoate was obtained (0.843 g, 3.11 mmol; 44% yield) as a light brown-gray solid. MS (+ESI): m/z 272.0 [M+H]⁺.

### Step 2: 4-Chloro-3-(pyridin-2-ylethynyl)benzoic acid

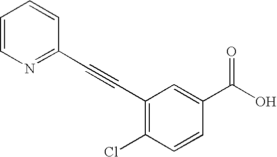

Methyl 4-chloro-3-(pyridin-2-ylethynyl)benzoate (0.413 g, 1.52 mmol) was dissolved in 6 mL of methanol. Aqueous 2N NaOH (1.52 mL, 3.05 mmol) was added, and the mixture was stirred 24 hours at room temperature. Aqueous 2N HCl (1.52 mL, 3.05 mmol) was added, and the mixture was stirred 5 minutes at room temperature. The mixture was evaporated to dryness to afford 4-chloro-3-(pyridin-2-ylethynyl)benzoic acid (0.580 g) as a light gray solid containing 2 equivalents of sodium chloride. This material was used as is for subsequent reactions. MS (+ESI): m/z 258.0 [M+H]⁺.

### Step 3: 2-[4-Chloro-3-(pyridin-2-ylethynyl)benzoyl]isoindoline

4-Chloro-3-(pyridin-2-ylethynyl)benzoic acid containing 2 equivalents of sodium chloride (0.030 g, 0.080 mmol) was dissolved in 0.8 mL dimethylformamide. N-(3-dimethylaminopropyl)-N′-ethyl-carbodiimide hydrochloride (EDCl, 0.019 g, 0.099 mmol) was added, followed by 1-hydroxy-7-azabenzotriazole (HOAt, 0.013 g, 0.096 mmol) and then isoindoline (0.011 mL, 0.097 mmol). Triethylamine (0.034 mL, 0.244 mmol) was added, and the mixture was stirred overnight at room temperature. LCMS analysis indicates about 10% of starting acid remains. More isoindoline (0.010 mL, 0.088 mmol) and EDCl (0.015 g, 0.078 mmol) were added, and the mixture stirred another 24 hours at room temperature. The mixture was then partitioned between ethyl acetate and water, and the aqueous layer was extracted with ethyl acetate. The combined organic phase was pumped dry, and was purified by prep HPLC using a Gilson reversed-phase HPLC with TFA modified water and acetonitrile as eluant. The solid obtained from the fractions containing the desired product was taken up in 0.7 mL methanol, and 2N HCl (0.100 mL, 0.200 mmol) was added. The mixture was stirred at room temperature for 5 minutes, and was then pumped dry to afford the HCl salt of 2-[4-chloro-3-(pyridin-2-ylethynyl)benzoyl]-isoindoline (0.014 g, 0.035 mmol; 44% yield) as a gray solid. MS (+ESI): m/z 359.1 [M+H]⁺.

### Example 61

### 2-[4-Chloro-3-(pyridin-2-ylethynyl)benzoyl]-1,2,3,4-tetrahydroisoquinoline

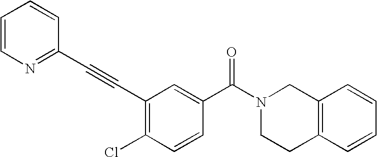

4-Chloro-3-(pyridin-2-ylethynyl)benzoic acid containing 2 equivalents of sodium chloride (0.033 g, 0.088 mmol) was dissolved in 0.8 mL dimethylformamide. N-(3-dimethylaminopropyl)-N′-ethyl-carbodiimide hydrochloride (EDCl, 0.020 g, 0.104 mmol) was added, followed by 1-hydroxy-7-azabenzotriazole (HOAt, 0.015 g, 0.110 mmol) and then 1,2,3,4-tetrahydroisoquinoline (0.012 mL, 0.098 mmol). Triethylamine (0.037 mL, 0.266 mmol) was added, and the mixture was stirred two days at room temperature. The mixture was then partitioned between ethyl acetate and water, and the aqueous layer was extracted with ethyl acetate. The combined organic phase was pumped dry, and was purified by prep HPLC using a Gilson reversed-phase HPLC with TFA modified water and acetonitrile as eluant. The solid obtained from the fractions containing the desired product was taken up in 0.7 mL methanol, and 2N HCl (0.100 mL, 0.200 mmol) was added. The mixture was stirred at room temperature for 5 minutes, and was then pumped dry to afford the HCl salt of 2-[4-chloro-3-(pyridin-2-ylethynyl)benzoyl]-1,2,3,4-tetrahydroisoquinoline (0.029 g, 0.078 mmol; 89% yield) as an ivory solid. MS (+ESI): m/z 373.1 [M+H]⁺.

A procedure similar to that of Examples 60 and 61, substituting the required cyclic secondary amine for isoindoline or tetrahydroisoquinoline, provides Examples 62-70. The compounds and their analytical data are shown in Table 2.

TABLE 2
Compounds Prepared According to the Procedure of Examples 60 and 61

| Example | Compound Name | MS |
|---|---|---|
| 62 | 7-{[4-chloro-3-(pyridin-2- | (+ESI): m/z 374.1 [M + H]⁺ |
| | ylethynyl)phenyl]carbonyl}-5,6,7,8-tetrahydro-1,7- | |
| | naphthyridine | |
| 63 | 2-{[4-chloro-3-(pyridin-2- | (+ESI): m/z 409.0 [M + H]⁺ |
| | ylethynyl)phenyl]carbonyl}-5,8-difluoro-1,2,3,4- | |
| | tetrahydroisoquinoline | |
| 64 | 2-{[4-chloro-3-(pyridin-2- | (+ESI): m/z 391.0 [M + H]⁺ |
| | ylethynyl)phenyl]carbonyl}-5-fluoro-1,2,3,4- | |
| | tetrahydroisoquinoline | |
| 65 | 6-{[4-chloro-3-(pyridin-2- | (+ESI): m/z 360.0 [M + H]⁺ |
| | ylethynyl)phenyl]carbonyl}-6,7-dihydro-5H- | |
| | pyrrolo[3,4-b]pyridine | |
| 66 | 5-{[4-chloro-3-(pyridin-2- | (+ESI): m/z 363.1 [M + H]⁺ |
| | ylethynyl)phenyl]carbonyl}-4,5,6,7-tetrahydro-1H- | |
| | pyrazolo[4,3-c]pyridine | |
| 67 | 5-{[4-chloro-3-(pyridin-2- | (+ESI): m/z 363.1 [M + H]⁺ |
| | ylethynyl)phenyl]carbonyl}-4,5,6,7-tetrahydro-1H- | |
| | imidazo[4,5-c]pyridine | |
| 68 | 6-{[4-chloro-3-(pyridin-2- | (+ESI): m/z 374.1 [M + H]⁺ |
| | ylethynyl)phenyl]carbonyl}-5,6,7,8-tetrahydro-1,6- | |
| | naphthyridine | |
| 69 | 5-chloro-2-{[4-chloro-3-(pyridin-2- | (+ESI): m/z 407.1 [M + H]⁺ |
| | ylethynyl)phenyl]carbonyl}-1,2,3,4- | |
| | tetrahydroisoquinoline | |
| 70 | 2-{[4-chloro-3-(pyridin-2- | (+ESI): m/z 391.1 [M + H]⁺ |
| | ylethynyl)phenyl]carbonyl}-7-fluoro-1,2,3,4- | |
| | tetrahydroisoquinoline | |

### Example 71

### 2-[4-Fluoro-3-(pyridin-2-ylethynyl)benzoyl]-1,2,3,4-tetrahydroisoquinoline

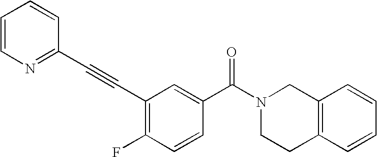

### Step 1: Methyl 4-fluoro-3-(pyridin-2-ylethynyl)benzoate

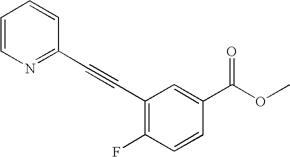

Methyl 3-bromo-4-fluorobenzoate (4.66 g, 20 mmol), 2-ethynylpyridine (3.2 mL, 31 mmol), and triethylamine (6.2 mL, 44.7 mmol) were dissolved in 100 mL of toluene and purged with nitrogen. The CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (40:1 CH₂Cl₂/EtOAc) to yield 2.0 g (39%) of the product as a brown solid. HRMS (+ESI): m/z 256.0769 [M+H]⁺.

### Step 2: 4-Fluoro-3-(pyridin-2-ylethynyl)benzoic acid

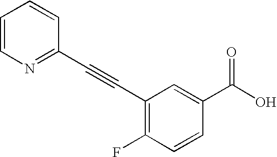

Methyl 4-fluoro-3-(pyridin-2-ylethynyl)benzoate (1.7 g, 6.6 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.24 g (78%) of the carboxylic acid as a tan solid. No additional purification of the carboxylic acid was required. HRMS (+ESI): m/z 242.0616 [M+H]⁺.

### Step 3: 2-[4-Fluoro-3-(pyridin-2-ylethynyl)benzoyl]-1,2,3,4-tetrahydroisoquinoline

4-Fluoro-3-(pyridin-2-ylethynyl)benzoic acid (48 mg, 0.2 mmol), tetrahydroisoquinoline (32 uL, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 57 mg (80%) of the product as a yellow foam. HRMS (+ESI): m/z 357.1399 [M+H]⁺.

A procedure similar to that of Example 71, substituting the required cyclic secondary amine for tetrahydroisoquinoline, provides Examples 72-74. The compounds and their analytical data are shown in Table 3.

TABLE 3
Compounds Prepared According to the Procedure of Example 71

| Example | COMPOUND NAME | MS |
|---|---|---|
| 72 | 7-chloro-2-[4-fluoro-3-(pyridin-2- | (+ESI): |
| | ylethynyl)benzoyl]- | m/z 391.1 [M + H]⁺ |
| | 1,2,3,4-tetrahydroisoquinoline | |
| 73 | 5-chloro-2-[4-fluoro-3-(pyridin-2- | (+ESI): |
| | ylethynyl)benzoyl]-1,2,3,4- | m/z 391.1 [M + H]⁺ |
| | tetrahydroisoquinoline | |
| 74 | 2-[4-fluoro-3-(pyridin-2- | (+ESI): |
| | ylethynyl)benzoyl]isoindoline | m/z 342.1 [M + H]⁺ |

### Example 75

### 2-[4-Ethoxy-3-(pyridin-2-ylethynyl)benzoyl]-1,2,3,4-tetrahydroisoquinoline

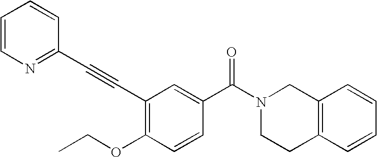

### Step 1: Methyl 4-ethoxy-3-iodobenzoate

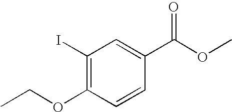

Methyl 4-hydroxy-3-iodobenzoate (2.78 g, 10 mmol) was dissolved in 20 mL of DMF and treated with Cs₂CO₃ (6.5 g, 20 mmol) and ethyliodide (1.0 mL, 12 mmol). The resulting suspension was stirred at room temperature overnight. The reaction mixture was subsequently diluted with EtOAc and washed with water (×2) and brine. The organic layer was dried (MgSO₄), filtered, and concentrated at reduced pressure to yield 3.0 g of a white solid. The crude material was used in the next step without additional purification. MS (+ESI): m/z 307.0 [M+H]⁺.

### Step 2: Methyl 4-ethoxy-3-(pyridin-2-ylethynyl)benzoate

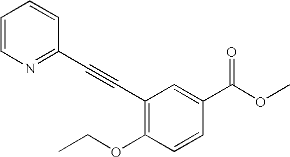

Crude methyl 4-ethoxy-3-iodobenzoate (10 mmol), 2-ethynylpyridine (1.6 mL, 15 mmol), and triethylamine (3.1 mL, 22 mmol) were dissolved in 50 mL of toluene and purged with nitrogen. The CuI (390 mg, 2 mmol) and Pd(Ph₃P)₂Cl₂ (1.45 g, 2 mmol) were added and the resulting suspension is stirred at 100 C for 6 hours. The reaction was concentrated at reduced pressure and purified by flash chromatography on silica (40:1 CH₂Cl₂/EtOAc) to yield 1.25 g (44% for 2 steps) of the product as a white solid. HRMS (+ESI): m/z 282.1124 [M+H]⁺.

### Step 3: 4-Ethoxy-3-(pyridin-2-ylethynyl)benzoic acid

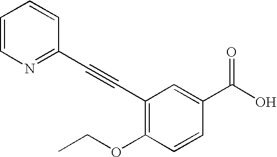

Methyl 4-ethoxy-3-(pyridin-2-ylethynyl)benzoate (1.1 g, 3.9 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 857 mg (82%) of the carboxylic acid as an off-white solid. No additional purification of the carboxylic acid is required. HRMS (+ESI): m/z 268.0969 [M+H]⁺.

### Step 4: 2-[4-Ethoxy-3-(pyridin-2-ylethynyl)benzoyl]-1,2,3,4-tetrahydroisoquinoline

4-Ethoxy-3-(pyridin-2-ylethynyl)benzoic acid (53 mg, 0.2 mmol), tetrahydroisoquinoline (32 uL, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 70 mg (92%) of the product as a tan foam. HRMS (+ESI): m/z 383.1755 [M+H]⁺.

A procedure similar to that of Example 75, substituting the required cyclic secondary amine for tetrahydroisoquinoline, provides Examples 76-78. The compounds and their analytical data are shown in Table 4.

TABLE 4
Compounds Prepared According to the Procedure of Example 75

| Example | Compound Name | MS |
|---|---|---|
| 76 | 7-chloro-2-[4-ethoxy-3-(pyridin-2- | (+ESI): |
| | ylethynyl)benzoyl]-1,2,3,4- | m/z 417.1 [M + H]⁺ |
| | tetrahydroisoquinoline | |
| 77 | 5-chloro-2-[4-ethoxy-3-(pyridin-2- | (+ESI): |
| | ylethynyl)benzoyl]-1,2,3,4- | m/z 417.1 [M + H]⁺ |
| | tetrahydroisoquinoline | |
| 78 | 2-[4-ethoxy-3-(pyridin-2- | (+ESI): |
| | ylethynyl)benzoyl]isoindoline | m/z 369.2 [M + H]⁺ |

### Example 79

### 2-{[4-(Cyclopropylmethoxy)-3-(pyridin-2-ylethynyl)phenyl]carbonyl}-1,2,3,4-tetrahydroisoquinoline

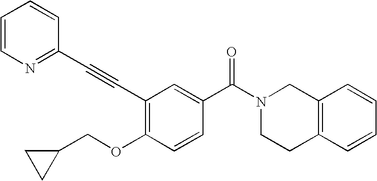

### Step 1: Methyl 4-(cyclopropylmethoxy)-3-iodobenzoate

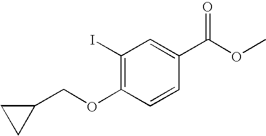

Methyl 4-hydroxy-3-iodobenzoate (2.78 g, 10 mmol) was dissolved in 20 mL of DMF and treated with Cs₂CO₃ (6.5 g, 20 mmol) and cyclopropylmethyl bromide (1.25 mL, 12 mmol). The resulting suspension was stirred at room temperature overnight. The reaction mixture was subsequently diluted with EtOAc and washed with water (×2) and brine. The organic layer was dried (MgSO₄), filtered, and concentrated at reduced pressure to yield 3.3 g of a pale yellow oil. The crude material was used in the next step without additional purification. MS (+ESI): m/z 333.0 [M+H]⁺.

### Step 2: Methyl 4-(cyclopropylmethoxy)-3-(pyridin-2-ylethynyl)benzoate

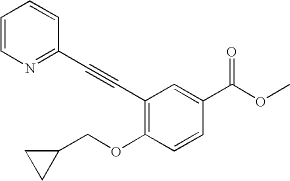

Crude methyl 4-(cyclopropylmethoxy)-3-iodobenzoate (10 mmol), 2-ethynylpyridine (1.6 mL, 15 mmol), and triethylamine (3.1 mL, 22 mmol) were dissolved in 50 mL of toluene and purged with nitrogen. Then, CuI (390 mg, 2 mmol) and Pd(Ph₃P)₂Cl₂ (1.45 g, 2 mmol) were added and the resulting suspension was stirred at 100° C. for 6 hours. The reaction was concentrated at reduced pressure and purified by flash chromatography on silica (CH₂Cl₂/EtOAc) to yield 1.52 g (50% for 2 steps) of the product as an oil. MS (+ESI): m/z 308.1 [M+H]⁺.

### Step 3: 4-(Cyclopropylmethoxy)-3-(pyridin-2-ylethynyl)benzoic acid

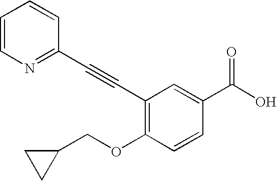

Methyl 4-(cyclopropylmethoxy)-3-(pyridin-2-ylethynyl)benzoate (1.5 g, 4.9 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.31 g (91%) of the carboxylic acid as a pale yellow solid. No additional purification of the carboxylic acid is required. HRMS (+ESI): m/z 294.1127 [M+H]⁺.

### Step 4: 2-{[4-(Cyclopropylmethoxy)-3-(pyridin-2-ylethynyl)phenyl]carbonyl}-1,2,3,4-tetrahydroisoquinoline

4-(Cyclopropylmethoxy)-3-(pyridin-2-ylethynyl)benzoic acid (59 mg, 0.2 mmol), tetrahydroisoquinoline (32 uL, 025 mmol), and triethylamine (84 uL, 0.6 mmol) were dissolved in 4 mL of CH₂Cl₂ and treated with HOBt (40 mg, 0.3 mmol) and EDC (58 mg, 0.3 mmol). The reaction was stirred at room temperature overnight and directly purified by flash chromatography on silica gel (CH₂Cl₂/EtOAc) to yield 65 mg (79%) of the product as an off-white solid. HRMS (+ESI): m/z 409.1909 [M+H]⁺.

### Example 80

### 2-{[4-(Cyclopropylmethoxy)-3-(pyridin-2-ylethynyl)phenyl]carbonyl}-2,3-dihydro-1H-isoindole

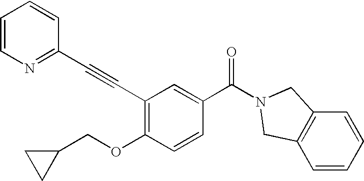

A procedure similar to that of Example 79, substituting isoindoline for tetrahydro-isoquinoline, provided 79 mg (100%) of (2-{[4-(cyclopropylmethoxy)-3-(pyridin-2-ylethynyl)phenyl]carbonyl}-2,3-dihydro-1H-isoindole. HRMS (+ESI): m/z 395.1751 [M+H]⁺.

### Example 81

### 2-[4-Methyl-3-(pyridin-2-ylethynyl)benzoyl]isoindoline

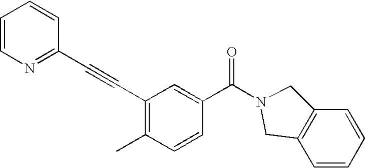

### Step 1: 2-[3-Iodo-4-methylbenzoyl]isoindoline

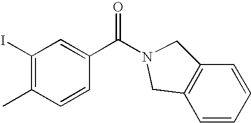

3-Iodo-4-methylbenzoic acid (262 mg, 1.0 mmol), isoindoline (284 uL, 1.5 mmol), and triethylamine (420 uL, 3.0 mmol) were dissolved in 10 mL of CH₂Cl₂ and treated with HOBt (203 mg, 1.5 mmol) and EDC (288 mg, 1.5 mmol). The reaction were stirred at room temperature overnight. The reaction mixture was concentrated, diluted with EtOAc, and washed with water, 1N HCl, and brine. The organic layer was dried (MgSO₄), filtered, and concentrated. The crude residue was purified by flash chromatography on silica gel (CH₂Cl₂/EtOAc) to yield 301 mg (83%) of the product as a tan solid. MS (+ESI): m/z 363.9 [M+H]⁺.

### Step 2: 2-[4-Methyl-3-(pyridin-2-ylethynyl)benzoyl]isoindoline2-[3-Iodo-4-methylbenzoyl]isoindoline

(109 mg, 0.3 mmol), 2-ethynylpyridine (45 uL, 0.45 mmol), and triethylamine (84 uL, 0.6 mmol) were dissolved in 3 mL of toluene and purged with nitrogen. The CuI (12 mg, 0.06 mmol) and Pd(Ph₃P)₂Cl₂ (42 mg, 0.06 mmol) were added and the resulting suspension was stirred at 100° C. for 6 hours. The reaction was concentrated at reduced pressure and purified by flash chromatography on silica (CH₂Cl₂/EtOAc) to yield 60 mg (39%) of the product as a brown solid. MS (+ESI): m/z 339.1 [M+H]⁺.

### Example 82

### 2-[4-Methyl-3-(pyridin-2-ylethynyl)benzoyl]-1,2,3,4-tetrahydroisoquinoline

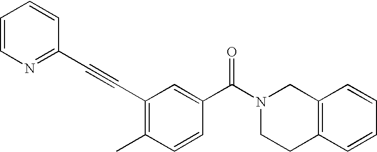

### Step 1: Methyl 4-methyl-3-(pyridin-2-ylethynyl)benzoate

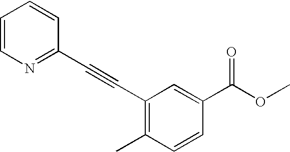

Methyl 3-iodo-4-methylbenzoate (5.52 g, 20 mmol), 2-ethynylpyridine (3.2 mL, 31 mmol), and triethylamine (6.2 mL, 44.7 mmol) were dissolved in 100 mL of toluene and purged with nitrogen. Then CuI (0.78 g, 3.9 mmol) and Pd(Ph₃P)₂Cl₂ (2.9 g, 4.1 mmol) were added and the resulting suspension was stirred at 100° C. for 6 hours. The reaction was concentrated at reduced pressure and purified by flash chromatography on silica (40:1 CH₂Cl₂/EtOAc) to yield 2.63 g (52%) of the product as a greenish solid. HRMS (+ESI): m/z 252.1019 [M+H]⁺.

### Step 2: 4-Methyl-3-(pyridin-2-ylethynyl)benzoic acid

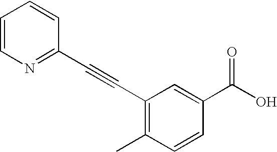

Methyl 4-methyl-3-(pyridin-2-ylethynyl)benzoate (2.2 g, 8.7 mmol) was dissolved in a mixture of THF (75 mL), MeOH (25 mL), and H₂O (25 mL) and treated with lithium hydroxide monohydrate (420 mg, 10 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 1.57 g (76%) of the carboxylic acid as a gray solid. No additional purification of the carboxylic acid was required. HRMS (+ESI): m/z 238.0865 [M+H]⁺.

### Step 3: 2-[4-Methyl-3-(pyridin-2-ylethynyl)benzoyl]-1,2,3,4-tetrahydroisoquinoline

4-Methyl-3-(pyridin-2-ylethynyl)benzoic acid (47 mg, 0.2 mmol), tetrahydroisoquinoline (32 uL, 0.25 mmol), and triethylamine (139 uL, 1.0 mmol) were dissolved in 3 mL of CH₂Cl₂ and treated with PyBOP (130 mg, 0.25 mmol). The reaction was stirred at room temperature overnight and directly purified by flash chromatography on silica gel (CH₂Cl₂/EtOAc) to yield 33 mg (47%) of the product as a colorless foam. HRMS (+ESI): m/z 353.1652 [M+H]⁺.

A procedure similar to that of Example 82, substituting the required cyclic secondary amine for tetrahydroisoquinoline, provides Examples 83-84. The compounds and their analytical data are shown in Table 5.

TABLE 5
Compounds Prepared According to the Procedure of Example 82

| Example | Compound Name | MS |
|---|---|---|
| 83 | 7-chloro-2-[4-methyl-3-(pyridin-2- | (+ESI): |
| | ylethynyl)benzoyl]-1,2,3,4- | m/z 387.1 [M + H]⁺ |
| | tetrahydroisoquinoline | |
| 84 | 5-chloro-2-[4-methyl-3-(pyridin-2- | (+ESI): |
| | ylethynyl)benzoyl]-1,2,3,4- | m/z 387.1 [M + H]⁺ |
| | tetrahydroisoquinoline | |

### Example 85

### 6-{[4-Methyl-3-(pyridin-2-ylethynyl)phenyl]carbonyl}-5,6,7,8-tetrahydro-1,6-naphthyridine

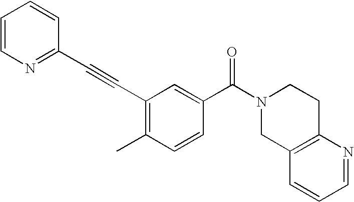

4-Methyl-3-(pyridin-2-ylethynyl)benzoic acid (24 mg, 0.1 mmol), 5,6,7,8-tetrahydro-1,6-naphthyridine dihydrochloride salt (26 mg, 0.125 mmol), and triethylamine (70 uL, 0.5 mmol) were dissolved in 2 mL of CH₂Cl₂ and treated with HOBt (17 mg, 0.125 mmol) and EDC (24 mg, 0.125 mmol). The reaction was stirred at room temperature overnight and directly purified by flash chromatography on silica gel (CH₂Cl₂/EtOAc) to yield 32 mg (90%) of the product as a tan solid. HRMS (+ESI): m/z 354.1606 [M+H]⁺.

A procedure similar to that of Example 85, substituting the required cyclic secondary amine for tetrahydroisoquinoline, provides Examples 86-91. The compounds and their analytical data are shown in Table 6.

TABLE 6
Compounds Prepared According to the Procedure of Example 85

| Example | Compound Name | MS |
|---|---|---|
| 86 | 7-{[4-Methyl-3-(pyridin-2- | (+ESI): m/z 354.1 [M + H]⁺ |
| | ylethynyl)phenyl]carbonyl}-5,6,7,8-tetrahydro- | |
| | 1,7-naphthyridine | |
| 87 | 5-{[4-methyl-3-(pyridin-2- | (+ESI): m/z 343.2 [M + H]⁺ |
| | ylethynyl)phenyl]carbonyl}-4,5,6,7-tetrahydro- | |
| | 3H-imidazo[4,5-c]pyridine | |
| 88 | 5-{[4-methyl-3-(pyridin-2- | (+ESI): m/z 343.2 [M + H]⁺ |
| | ylethynyl)phenyl]carbonyl}-4,5,6,7-tetrahydro- | |
| | 1H-pyrazolo[4,3-c]pyridine | |
| 89 | 2-methyl-5-{[4-methyl-3-(pyridin-2- | (+ESI): m/z 374.1 [M + H]⁺ |
| | ylethynyl)phenyl]carbonyl}-4,5,6,7- | |
| | tetrahydro[1,3]thiazolo[5,4-c]pyridine | |
| 90 | 2-methyl-5-{[4-methyl-3-(pyridin-2- | (+ESI): m/z 358.2 [M + H]⁺ |
| | ylethynyl)phenyl]carbonyl}-4,5,6,7- | |
| | tetrahydro[1,3]oxazolo[4,5-c]pyridine | |
| 91 | 6-{[4-methyl-3-(pyridin-2- | (+ESI): m/z 340.2 [M + H]⁺ |
| | ylethynyl)phenyl]carbonyl}-6,7-dihydro-5H- | |
| | pyrrolo[3,4-b]pyridine | |

### Example 92

### 6-{[3-(Pyridin-2-ylethynyl)-4-(trifluoromethyl)phenyl]carbonyl}-6,7-dihydro-5H-pyrrolo[3,4-b]pyridine, di-TFA salt

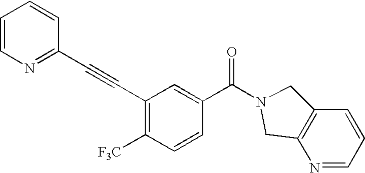

### Step 1: Methyl 3-amino-4-(trifluoromethyl)benzoate, HCl salt

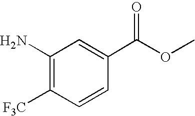

A solution of 3-amino-4-(trifluoromethyl)benzoic acid (10 g, 48.8 mmol) and concentrated HCl (36%, 5.5 mL) in methanol (42 mL) was heated at 70° C. for 10 hours. After the reaction was complete, the reaction mixture was cooled down and concentrated in vacuo to afford methyl 3-amino-4-(trifluoromethyl)benzoate, HCl salt as a white solid (8.9 g, 34.8 mmol; 71% yield). MS (+ESI): m/z 220.0 [M+H]⁺.

### Step 2: Methyl 3-iodo-4-(trifluoromethyl)benzoate

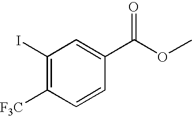

A solution of sodium nitrite (1.34 g 19.3 mmol) in water (7.0 mL) was added dropwise to a rapidly stirred suspension of methyl 3-amino-4-(trifluoromethyl)benzoate, HCl salt (4.5 g, 17.5 mmol) from step 1 in 6 N aqueous HCl (11 mL) at −5 to 0° C. over a period of five min. After the reaction was stirred at −5° C. for 30 min., a solution of potassium iodide (2.9 g, 17.5 mmol) in water (6.0 mL) and a small crystal of iodine was added slowly to the diazonium chloride formed in the reaction suspension. The resulting dark red solution was allowed to warm to room temperature and was heated at 90° C. for one hour. The reaction mixture was extracted with ethyl acetate. The collected ethyl acetate extracts were washed with water. Separation and evaporation afforded methyl 3-iodo-4-(trifluoromethyl)benzoate as a dark brown solid (5.2 g, 15.8 mmol; 90% yield). MS (+ESI): m/z 330.0 [M+H]⁺.

### Step 3: Methyl 3-(pyridin-2-ylethynyl)-4-(trifluoromethyl)benzoate

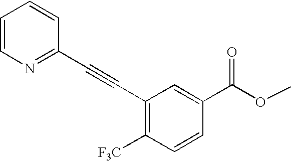

A mixture of methyl 3-iodo-4-(trifluoromethyl)benzoate (3 g, 9.1 mmol) from step 2,2-ethynylpyridine (1.42 mL, 13.6 mmol), dichlorobistriphenylphosphine palladium(II) (1.28 g, 1.8 mmol), copper iodide (0.36 g, 1.82 mmol) and triethylamine (2.6 mL, 18.2 mmol) in toluene (46 mL) was stirred at 100° C. for six hours. The reaction mixture was monitored by LC-MS. After the reaction was complete, the reaction mixture was then allowed to cool down to room temperature. The reaction mixture was concentrated to yield a semi-solid residue. This residue was dissolved in ethyl acetate and the un-dissolved dark solid was removed by filtration. The ethyl acetate filtrate was washed with water and brine, dried over magnesium sulfate, filtered, and concentrated in vacuo to provide a brown crude solid. This material was purified by flash chromatography on SiO₂ (gradient elution using EtOAc/hexane) to yield the title compound as a brown solid (1.5 g, 4.9 mmol; 54% yield). MS (+ESI): m/z 306.0 [M+H]⁺.

### Step 4: 3-(Pyridin-2-ylethynyl)-4-(trifluoromethyl)benzoic acid

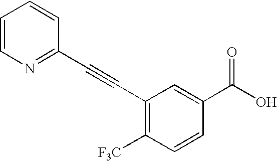

A 1.0 N solution of aqueous sodium hydroxide (7.3 mL, 7.3 mmol) was added to a solution of methyl 3-(pyridin-2-ylethynyl)-4-(trifluoromethyl)benzoate (1.1 g, 3.7 mmol) from step 3 in a mixed solvent of methanol and tetrahydrofuran (1:1; 20 mL) with stirring at room temperature. The reaction was complete in six hours. The reaction was acidified with 2.0 N aqueous HCl (3.7 mL, 7.3 mmol) to pH=1. The suspended mixture was filtered and evaporated to afford a light brown solid (1.5 g, 3.7 mmol; 100% yield) containing 2 eq. of sodium chloride, which was used for the next reaction without any further purification. MS (+ESI): m/z 292.0 [M+H]⁺.

### Step 5: 6-{[3-(Pyridin-2-ylethynyl)-4-(trifluoromethyl)phenyl]carbonyl}-6,7-dihydro-5H-pyrrolo[3,4-b]pyridine, TFA salt

Triethylamine (0.084 mL, 0.6 mmol) was added to a mixture of 3-(pyridin-2-ylethynyl)-4-(trifluoromethyl)benzoic acid (containing 2 eq. of sodium chloride, 61.2 mg, 0.15 mmol) from step 4, 1-[3-(dimethylamino)propyl]-3-ethylcarbodiimide hydrochloride (36 mg, 0.18 mmol), 1-hydroxy-7-azabenzotriazole (24 mg, 0.18 mmol) and 6,7-dihydro-5H-pyrrolo[3,4-b]pyridine (di-HCl salt, 33 mg, 0.17 mmol) in dichloromethane (2.1 mL) with stirring at room temperature under an atmosphere of nitrogen. The reaction mixture was stirred at room temperature overnight. The reaction was quenched with small amount of water. Solvents were removed and the residue was purified by reverse phase Gilson HPLC (mobile phase: 20-100% acetonitrile (with 0.075% TFA) in water (with 0.075% TFA) to afford the title compound as TFA salt (75 mg, 0.14 mmol; 93% yield). MS (+ESI): m/z 394.1 [M+H]⁺.

A procedure similar to that of Example 92, substituting the required cyclic secondary amine for 6,7-dihydro-5H-pyrrolo[3,4-b]pyridine, provides Examples 93-100. The compounds and their analytical data are shown in Table 7.

TABLE 7
Compounds Prepared According to the Procedure of Example 92

| Example | Compound Name | MS |
|---|---|---|
| 93 | 2-{[3-(Pyridin-2-ylethynyl)-4- | (+ESI): m/z 407.1 [M + H]⁺ |
| | (trifluoromethyl)phenyl]carbonyl}-1,2,3,4- | |
| | tetrahydroisoquinoline, TFA salt | |
| 94 | 7-Chloro-2-{[3-(pyridin-2-ylethynyl)-4- | (+ESI): m/z 441.0 [M + H]⁺ |
| | (trifluoromethyl)phenyl]carbonyl}-1,2,3,4- | |
| | tetrahydroisoquinoline, TFA salt | |
| 95 | 5-Chloro-2-{[3-(pyridin-2-ylethynyl)-4- | (+ESI): m/z 441.0 [M + H]⁺ |
| | (trifluoromethyl)phenyl]carbonyl}-1,2,3,4- | |
| | tetrahydroisoquinoline, TFA salt | |
| 96 | 2-{[3-(Pyridin-2-ylethynyl)-4- | (+ESI): m/z 393.1 [M + H]⁺ |
| | (trifluoromethyl)phenyl]carbonyl}-2,3-dihydro-1H- | |
| | isoindole, TFA salt | |
| 97 | 3-Bromo-6-{[3-(pyridin-2-ylethynyl)-4- | (+ESI): m/z 486.0 [M + H]⁺ |
| | (trifluoromethyl)phenyl]carbonyl}-5,6,7,8- | |
| | tetrahydro-1,6-naphthyridine, di-TFA salt | |
| 98 | 6-{[3-(Pyridin-2-ylethynyl)-4- | (+ESI): m/z 408.1 [M + H]⁺ |
| | (trifluoromethyl)phenyl]carbonyl}-5,6,7,8- | |
| | tetrahydro-1,6-naphthyridine, di-TFA salt | |
| 99 | 7-{[3-(Pyridin-2-ylethynyl)-4- | (+ESI): m/z 408.1 [M + H]⁺ |
| | (trifluoromethyl)phenyl]carbonyl}-5,6,7,8- | |
| | tetrahydro-1,7-naphthyridine, di-TFA salt | |
| 100 | 5-{[3-(Pyridin-2-ylethynyl)-4- | (+ESI): m/z 397.1 [M + H]⁺ |
| | (trifluoromethyl)phenyl]carbonyl}-4,5,6,7- | |
| | tetrahydro-1H-pyrazolo[4,3-c]pyridine, di-TFA salt | |
| 101 | 5-{[3-(Pyridin-2-ylethynyl)-4- | (+ESI): m/z 397.1 [M + H]⁺ |
| | (trifluoromethyl)phenyl]carbonyl}-4,5,6,7- | |
| | tetrahydro-1H-imidazo[4,5-c]pyridine, di-TFA salt | |
| 102 | 2-{[3-(Pyridin-2-ylethynyl)-4- | (+ESI): m/z 394.1 [M + H]⁺ |
| | (trifluoromethyl)phenyl]carbonyl}-2,3-dihydro-1H- | |
| | pyrrolo[3,4-c]pyridine, di-TFA salt | |

### Example 103

### 2-[3-(Pyridin-2-ylethynyl)benzoyl]isoindoline

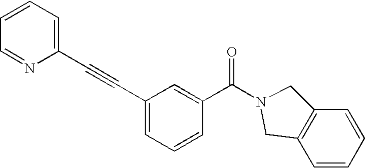

### Step 1: 2-[3-Iodobenzoyl]isoindoline

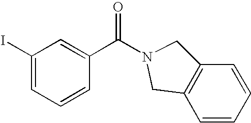

3-Iodobenzoic acid (248 mg, 1.0 mmol), isoindoline (284 uL, 1.5 mmol), and triethylamine (420 uL, 3.0 mmol) were dissolved in 10 mL of CH₂Cl₂ and treated with HOBt (203 mg, 1.5 mmol) and EDC (288 mg, 1.5 mmol). The reaction was stirred at room temperature overnight. The reaction mixture was concentrated, diluted with EtOAc, and washed with water, 1N HCl, and brine. The organic layer was dried (MgSO₄), filtered, and concentrated. The crude residue was purified by flash chromatography on silica gel (CH₂Cl₂/EtOAc) to yield 304 mg (87%) of the product as a tan solid. MS (+ESI): m/z 349.9 [M+H]⁺.

### Step 2: 2-[3-(Pyridin-2-ylethynyl)benzoyl]isoindoline

2-[3-Iodobenzoyl]isoindoline (105 mg, 0.3 mmol), 2-ethynylpyridine (45 uL, 0.45 mmol), and triethylamine (84 uL, 0.6 mmol) were dissolved in 3 mL of toluene and purged with nitrogen. Then CuI (12 mg, 0.06 mmol) and Pd(Ph₃P)₂Cl₂ (42 mg, 0.06 mmol) were added and the resulting suspension was stirred at 100° C. for 6 hours. The reaction was concentrated at reduced pressure and purified by flash chromatography on silica (CH₂Cl₂/EtOAc) to yield 72 mg (74%) of the product as a brown solid. MS (+ESI): m/z 325.0 [M+H]⁺.

### Example 104

### 2-{4-Methoxy-3-[(3-methoxyphenyl)ethynyl]benzoyl}isoindoline

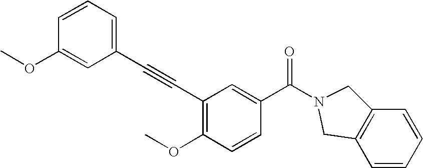

### Step 1: 3-Iodo-4-methoxybenzoic acid

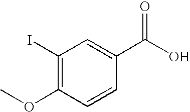

Methyl 3-iodo-4-methoxybenzoate (2.92 g, 10 mmol) was dissolved in a mixture of THF (80 mL), MeOH (10 mL), and H₂O (20 mL) and treated with lithium hydroxide monohydrate (840 mg, 20 mmol). The reaction was stirred at room temperature overnight and then concentrated at reduced pressure. The remaining residue was diluted with 50 mL of H₂O and acidified to pH 4.0 with 1N HCl. The resulting precipitate was collected by suction filtration. The collected precipitate was dried in a vacuum oven at 50° C. for 3 hours to yield 2.77 g (76%) of the carboxylic acid as a white solid. No additional purification of the carboxylic acid was required.

### Step 2: 2-(3-Iodo-4-methoxybenzoyl)isoindoline

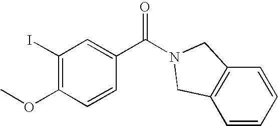

A 50 mL, round-bottom flask equipped with a magnetic stirbar was charged with 3-iodo-4-methoxybenzoic acid (500 mg, 1.97 mmol), HOBT (482 mg, 3.15 mmol) and EDC (567 mg, 2.96 mmol). The reagents were dissolved in 15 mL CH₂Cl₂ and the solution was allowed to stir for 15 min. Subsequently, isoindoline (0.447 mL, 3.94 mmol) and TEA (0.819 mL, 5.91 mmol) were added to the reaction vessel and the solution was allowed to stir for 5 h. Upon completion, the solvent was removed under reduced pressure and the residue was purified via flash chromatography on silica gel to afford 490 mg (65%) of the title compound.

### Step 3: 2-{4-Methoxy-3-[(3-methoxyphenyl)ethynyl]benzoyl}isoindoline

A solution consisting of 2-(3-iodo-4-methoxybenzoyl)isoindoline (50 mg, 0.132 mmol), 3-ethynylanisole (52 mg, 0.396 mmol), CuI (5 mg, 0.026 mmol), Pd(PPh₃)₂Cl₂ (18 mg, 0.026) and TEA (0.055 mL, 0.396 mmol) in 7.5 mL of toluene was stirred under nitrogen overnight in a 40 mL vial at 100° C. Upon cooling to rt, the crude mixture was filtered through a plug of silica gel and washed through with EtOAc. The solvents were then removed under reduced pressure and the residue was purified by flash chromatography on silica gel (20:1 CH₂Cl₂/MeOH) to afford 35 mg (69%) of the title compound as an off-white solid. ¹H NMR (400 MHz, CDCl₃): consistent; MS (ES⁺) m/z 384.1 (M+H).

A procedure similar to that of Example 104, substituting the required acetylene for 3-ethynylanisole, provides Examples 105-121. The compounds and their analytical data are shown in Table 8.

TABLE 8
Compounds Prepared According to the Procedure of Example 104

| Example | COMPOUND NAME | MS |
|---|---|---|
| 105 | 2-{3-[(2,4-difluorophenyl)ethynyl]-4- | (ES) m/z 390.0 (M + H) |
| | methoxybenzoyl}isoindoline | |
| 106 | 2-{3-[(4-fluoro-3-methylphenyl)ethynyl]-4- | (ES) m/z 386.0 (M + H) |
| | methoxybenzoyl}isoindoline | |
| 107 | 2-(4-methoxy-3-{[2- | (ES) m/z 422.0 (M + H) |
| | (trifluoromethyl)phenyl]ethynyl}benzoyl)isoindoline | |
| 108 | 2-{4-methoxy-3-[(2- | (ES) m/z 384.1 (M + H) |
| | methoxyphenyl)ethynyl]benzoyl}isoindoline | |
| 109 | 2-[3-(cyclohex-1-en-1-ylethynyl)-4- | (ES) m/z 358.1 (M + H) |
| | methoxybenzoyl]isoindoline | |
| 110 | 2-[4-methoxy-3-(pyridin-3- | (ES) m/z 355.1 (M + H) |
| | ylethynyl)benzoyl]isoindoline | |
| 111 | 2-{3-[(2-chlorophenyl)ethynyl]-4- | (ES) m/z 388.0 (M + H) |
| | methoxybenzoyl}isoindoline | |
| 112 | 4-{[5-(1,3-dihydro-2H-isoindol-2-ylcarbonyl)-2- | (ES) m/z 379.1 (M + H) |
| | methoxyphenyl]ethynyl}benzonitrile | |
| 113 | 3-{[5-(1,3-dihydro-2H-isoindol-2-ylcarbonyl)-2- | (ES) m/z 370.1 (M + H) |
| | methoxyphenyl]ethynyl}phenol | |
| 114 | 2-{3-[(3,5-dimethoxyphenyl)ethynyl]-4- | (ES) m/z 414.1 (M + H) |
| | methoxybenzoyl}isoindoline | |
| 115 | 2-[4-methoxy-3- | (ES) m/z 354.1 (M + H) |
| | (phenylethynyl)benzoyl]isoindoline | |
| 116 | 2-{4-methoxy-3-[(3- | (ES) m/z 368.1 (M + H) |
| | methylphenyl)ethynyl]benzoyl}isoindoline | |
| 117 | 2-{3-[(4-fluorophenyl)ethynyl]-4- | (ES) m/z 372.1 (M + H) |
| | methoxybenzoyl}isoindoline | |
| 118 | 2-{4-methoxy-3-[(4- | (ES) m/z 384.1 (M + H) |
| | methoxyphenyl)ethynyl]benzoyl}isoindoline | |
| 119 | 2-{3-[(3-chlorophenyl)ethynyl]-4- | (ESI) m/z 388.1 (M + H) |
| | methoxybenzoyl}isoindoline | |
| 120 | 2-{4-methoxy-3-[(6-methylpyridin-2- | (ESI) m/z 369.2 (M + H) |
| | yl)ethynyl]benzoyl}isoindoline | |
| 121 | 2-{4-methoxy-3-[(6-methylpyridin-2- | (ESI) m/z 383.2 (M + H) |
| | yl)ethynyl]benzoyl}-1,2,3,4-tetrahydroisoquinoline | |

### Example 122

### (4-(difluoromethoxy)-3-(pyridin-2-ylethynyl)phenyl)(5H-pyrrolo[3,4-b]pyridin-6(7H)-yl)methanone

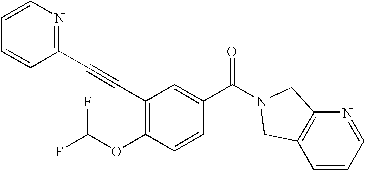

### Step 1: Methyl 4-(difluoromethoxy)-3-iodobenzoate

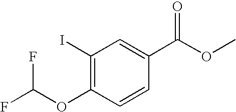

A cold solution of difluoroiodomethane (5.0 g, 28.0 mmol) in DMF (15 mL) was added to a stirred suspension of potassium carbonate (5.2 g, 37.4 mmol) and methyl 4-hydroxy-3-iodobenzoate (5.4 g, 97%, 18.7 mmol) in DMF (65 mL) at 0° C. under an atmosphere of nitrogen. After the reaction was stirred at 0° C. for 30 min., the reaction mixture was stirred at room temperature for 2.5 hours. After the reaction was completed, solids were removed by filtration and the filtrate was concentrated to a semi-solid residue. This residue was purified by flash chromatography on SiO₂ (gradient elution using EtOAc/hexane 15/85) to yield the title compound as a white solid (5.0 g, 81% yield).

### Step 2: Methyl 4-(difluoromethoxy)-3-(pyridin-2-ylethynyl)benzoate

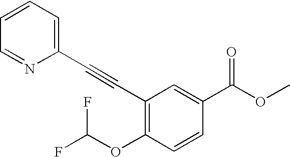

A mixture of methyl 4-(difluoromethoxy)-3-iodobenzoate (2 g, 6.1 mmol) from step 1,2-ethynylpyridine (0.94 mL, 9.2 mmol), dichlorobistriphenylphosphine palladium(II) (0.86 g, 1.2 mmol), copper iodide (0.23 g, 1.2 mmol) and triethylamine (1.7 mL, 12.2 mmol) in toluene (30 mL) was stirred at 100° C. under an atmosphere of nitrogen for six hours. After the reaction was complete, the reaction mixture was concentrated to yield a semi-solid residue. This residue was purified by flash chromatography on SiO₂ (gradient elution using EtOAc/hexane 20/80) to yield the title compound as a white solid (1.47 g, 80% yield).

### Step 3: 4-(Difluoromethoxy)-3-(pyridin-2-ylethynyl)benzoic acid

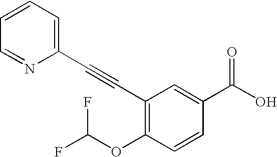

A 1.0 N solution of aqueous sodium hydroxide (9.6 mL, 9.6 mmol) was added to a solution of methyl 4-(difluoromethoxy)-3-(pyridin-2-ylethynyl)benzoate (1.5 g, 4.8 mmol) from step 2 in a mixed solvent of methanol and tetrahydrofuran (1:1; 26 mL) with stirring at room temperature. The reaction was complete in three hours. The reaction mixture was acidified with 2.0 N aqueous HCl (5.0 mL, 10.0 mmol) to pH=1. The suspended mixture was evaporated to afford a grey solid (1.84 g, 95% yield) containing two equivalents of sodium chloride, which was used for the next reaction without any further purification.

### STEP 4: (4-(difluoromethoxy)-3-(pyridin-2-ylethynyl)phenyl)(5H-pyrrolo[3,4-b]pyridin-6(7H)-yl)methanone, HCl salt

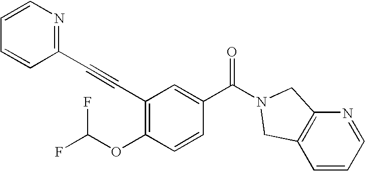

Triethylamine (2.3 mL, 16.7 mmol) was added to a mixture of 4-(difluoromethoxy)-3-(pyridin-2-ylethynyl)benzoic acid containing two equivalents of sodium chloride (1.7 g, 4.2 mmol) from step 3, 1-[3-(dimethylamino)propyl]-3-ethylcarbodiimide hydrochloride (1.1 g, 5.5 mmol), 1-hydroxy-7-azabenzotriazole (0.74 g, 5.5 mmol) and 6,7-dihydro-5H-pyrrolo[3,4-b]pyridine (di-HCl salt, 0.97 g, 5.0 mmol) in dichloromethane (64 mL) with stirring at room temperature under an atmosphere of nitrogen. The reaction mixture was stirred at room temperature overnight. The reaction was quenched with small amount of water. The solvents were removed and the residue was purified by flash chromatography on SiO₂ (gradient elution using dichloromethane/methanol 95/5) to afford a semi-solid, which was dissolved in methanol (50 mL). Aqueous HCl (2.0 N, 5.0 mL) was added to this methanol solution. The mixture was then stirred at room temperature for 20 min. Evaporation yielded a semi-solid, which was dried in vacuo at 50° C. for 6 hours to afford the title compound as a light green solid (1.76 g, 91% yield). MS (+ESI): m/z 392.1 [M+H]⁺.

### Determination of Activity of Compounds

### Biological Test Procedures:

### Procedure Number: 10710

TITLE: [³H]-MPEP binding HEK-293 cell membranes expressing a rat mGluR5 receptor.
PURPOSE: To identify high affinity compounds at the rat mGluR5 receptor based on their ability to displace the mGluR5 negative allosteric modulator, MPEP.

### Materials and Methods:

mGluR5 expressing HEK-293 cells are scraped off the plate, transferred to centrifuge tubes, and washed twice by centrifugation (2000 rpm for 10 min., 4° C.) in buffer (50 mM Tris pH 7.5). The resulting pellets are aliquoted and placed at −80° C. On the day of assay, the cells are thawed on ice, and resuspended in buffer. The binding assay is performed in a 96 well microtiter plate in a total volume of 250 μL. Non-specific binding is determined in the presence of 10 μM MPEP. Binding reaction includes a final radioligand [³H]-MPEP concentration of 4 nM and 12-25 ug membrane protein per well. Following a 60 minute incubation at room temperature, the reaction is terminated by the addition of ice cold buffer and rapid filtration through a GF/B filter presoaked for 30 minutes in 0.5% PEI. Compounds are initially tested in a single point assay to determine percent inhibition at 10 μM. Subsequently, Ki values are determined for compounds defined to be active.

### Analysis of Results:

% inhibition and Ki values generated by GraphPad Prism and Excel Fit. IC₅₀ values are calculated using GraphPad by fitting to a 1 or 2 site-binding model. Ki values are calculated from the apparent IC₅₀ values using the Cheng-Prussof equation:

$$Ki = \frac{{IC}_{50}}{1 + ( [ L ] / Kd )}$$

where [L]=concentration of free radioligand and Kd=dissociation constant of radioligand for the receptor.
TITLE: Identification of metabotropic glutamate receptor 5 antagonists using a stable, sensitive fluorescence-based screening system
PURPOSE: The aim of the research presented here was to develop a robust functional assay to measure changes in intracellular calcium, with the goal of identifying potent mGluR5 antagonists.

### Materials and Methods:

### FLIPR Calcium 3 Assay Kit Components

Each kit contains the following components.

| | |
|---|---|
| R8091 | 10 vials Component A |
| (Explorer | 1 bottle Component B (ready to use HBSS buffer - 1× Hank's |
| Kit) | Balanced Salt solution with 20 mM Hepes buffer, pH 7.4). |
| | Sufficient for ten 96- or 384-well plates. |
| | Each vial is sufficient for assaying one |
| | 96- or 384-well plate. |
| R8090 | 10 vials, sufficient for one hundred 96- or 384-well |
| (Bulk Kit) | plates. Each vial is sufficient for assaying |
| | ten 96- or 384-well plates. |
| R8108 | 2 vials, sufficient for one hundred 96-well or 384-well |
| (Express | plates. Each vial is sufficient for assaying fifty |
| Kit) | 96- or 384-well plates. |

additional materials needed but not included in kit

| | |
|---|---|
| HBSS Buffer (1× Hank's | 10X Hank's Balanced Salt Solution |
| Balanced Salt | (#14065-056, Gibco or equivalent) |
| solution with 20 mM Hepes buffer) | 1M Hepes buffer solution (#9319, |
| | Irvine Scientific or equivalent) |
| | Water for cell culture (# 9312, Irvine |
| | Scientific or equivalent) |
| Probenecid (inhibitor for the anion- | Sigma or other chemical suppliers |
| exchange protein) may be | |
| required with some cell lines. | |

### Storage & Handling

Store the FLIPR Calcium 3 Assay Kit at −20° C. Under these conditions the reagents are stable for six months in the original packaging. After formulation, the Loading Buffer is stable for up to eight hours at room temperature. Aliquots can be frozen and stored for up to 5 days without loss of activity.

### FLIPR Calcium 3 Assay Kit Experimental Protocol

### Cell Handling

The FLIPR Calcium 3 Assay Kit is designed to work with many cell types, both adherent and non-adherent. Optimal cell conditions for the FLIPR Calcium 3 Assay Kit require the creation of a confluent cell monolayer before placing the plates in FLIPR or FlexStation. In general, we start with 50,000 mGluR5 HEK-293 cells/well for a 96-well plate. For adherent cells, seeding cells overnight with a plating volume of 100 μL/well worked for 96-well plates.

### Preparation of Loading Buffer

The following procedure is designed for preparation of the loading buffer per vial of the Explorer Kit (R8091), the Bulk Kit (R8091), or the Express Kit (R8108). To prepare the 1×HBSS Buffer (for the Bulk Kit and the Express Kit only, Explorer Kit contains ready to use solution to 880 mL cell culture treated water. Remove one vial of FLIPR Calcium 3 Assay reagent and equilibrate to room temperature. Dissolve contents of vial completely by adding 10 mL (for Explorer kit and Bulk kit), 20 mL (for Express Kit) of 1×HBSS Buffer. Mix by repeated pipetting or vortexing until the contents are completely dissolved. Prepare the Loading Buffer by diluting the vial mixture in 1× Reagent Buffer. Multiple washes of the vial are necessary to completely transfer the contents.

Note: if cells require probenecid, then a stock solution should be prepared fresh and added fresh to the Loading Buffer at a final in-well working concentration of 2.5 mM. Do not store frozen aliquots of Loading Buffer with probenecid and always add fresh probenecid the day of the experiment.

### Loading the Cells Using Loading Buffer

Remove cell plates from the incubator. Remove the supernatant medium. Add 180 ul of Loading Buffer to each well (for 96-well plates). HBSS buffer component B) pipette 100 mL of 10× Hank's Balanced Salt Solution and 20 mL of 1M Hepes buffer

Note: Although Molecular Devices does not recommend washing cells before dye loading, growth medium and serum factors can be washed away before adding the Loading Buffer, provided residual volumes after the wash step are as described.

Incubate cell plates for 1 hour at 37° C. and then keep the plates at room temperature until used. Do NOT wash the cells after dye loading.

### Running the Calcium 3 Assay on FLIPR

After incubation, transfer the plates directly to FLIPR and begin the calcium assay as described in the FLIPR system manual. For a signal test, a starting average count of ˜10,000 is recommended. Recommended experimental setup parameters are as follows. Faster addition speeds can lead to better mixing of compounds and lower signal variance across the plate.

| Parameters | 96-well plate |
|---|---|
| Addition Volume (μL) | 20 |
| Compound Concentration (Fold) | 10X |
| Addition Speed (μL/sec) | 50-100 |
| Adherent cells | |

### Running the Calcium 3 Assay on FlexStation

Experimental setup parameters are as follows.

| Parameters | |
|---|---|
| Excitation Wavelength (nm) | 485 |
| Emission Wavelength (nm) | 525 |
| Emission Cut-off (nm) | 515 |
| Pipette Height (μL) | 150 |
| Transfer Volume (μL) | 20 |
| Compound Concentration (Fold) | 10X |
| Addition Speed (Rate) | 4-6 |
| Adherent Cells | |

After incubation, transfer the assay plate directly to the FlexStation assay plate carriage and run the assay.

### Determination of Antagonist Activity

Test or reference compound is added to the 96-well plate (manually). After 10-15 min, agonist (e.g. L-Glutamate) at EC₉₀ concentration (˜50 uM final concentration; 20 ul) is added to each well via automated FLIPR or FlexStation. Relative Fluorescence Units (RFUs) are recorded over a given time period (stated above) depending on the platform system used.

### Analysis of Results:

For antagonists, results are expressed as % inhibition of glutamate stimulated Calcium levels. IC₅₀ values were calculated by Prism GraphPad using a 4-parameter logistic equation. IC₅₀ values are calculated using GraphPad by fitting to a 1 site-model.

| | | mGluR5 | mGluR5 |
|---|---|---|---|
| | | Ligand | FLIPR MP |
| | | Binding | Antagonist |
| | | DR Ki | DR IC50 |
| EXAMPLE | COMPOUND NAME | (uM) | (uM) |
| 1 | 2-[4-methoxy-3-(pyridin-2- | 0.004 | 0.01 |
| | ylethynyl)benzoyl]isoindoline | | |
| 2 | 2-[4-methoxy-3-(pyridin-2-ylethynyl)benzoyl]- | 0.003 | 0.024 |
| | 1,2,3,4-tetrahydroisoquinoline | | |
| 3 | 5-fluoro-2-[4-methoxy-3-(pyridin-2- | 0.002 | 0.044 |
| | ylethynyl)benzoyl]-1,2,3,4-tetrahydroisoquinoline | | |
| 4 | 2-[4-methoxy-3-(pyridin-2-ylethynyl)benzoyl]-5- | 0.0004 | 0.134 |
| | (trifluoromethyl)-1,2,3,4-tetrahydroisoquinoline | | |
| 5 | 2-[4-methoxy-3-(pyridin-2-ylethynyl)benzoyl]-8- | 0.002 | 0.151 |
| | (trifluoromethyl)-1,2,3,4-tetrahydroisoquinoline | | |
| 6 | 5,8-difluoro-2-[4-methoxy-3-(pyridin-2- | 0.002 | 0.052 |
| | ylethynyl)benzoyl]-1,2,3,4-tetrahydroisoquinoline | | |
| 7 | 5,7-dichloro-2-[4-methoxy-3-(pyridin-2- | NT | 0.176 |
| | ylethynyl)benzoyl]-1,2,3,4-tetrahydroisoquinoline | | |
| 8 | 7-chloro-2-[4-methoxy-3-(pyridin-2- | 0.001 | 0.016 |
| | ylethynyl)benzoyl]-1,2,3,4-tetrahydroisoquinoline | | |
| 9 | 7-fluoro-2-[4-methoxy-3-(pyridin-2- | 0.003 | 0.023 |
| | ylethynyl)benzoyl]-1,2,3,4-tetrahydroisoquinoline | | |
| 10 | 5-chloro-2-[4-methoxy-3-(pyridin-2- | 0.001 | 0.05 |
| | ylethynyl)benzoyl]-1,2,3,4-tetrahydroisoquinoline | | |
| 11 | 2-[4-methoxy-3-(pyridin-2-ylethynyl)benzoyl]- | 0.011 | 0.13 |
| | 1,2,3,4-tetrahydroisoquinoline-7-carbonitrile | | |
| 12 | 2-[4-methoxy-3-(pyridin-2-ylethynyl)benzoyl]- | 0.008 | >1.000 |
| | 2,3,4,5-tetrahydro-1H-pyrido[4,3-b]indole | | |
| 13 | 5-[4-methoxy-3-(pyridin-2-ylethynyl)benzoyl]- | 0.005 | 0.044 |
| | 4,5,6,7-tetrahydrothieno[3,2-c]pyridine | | |
| 14 | 3-bromo-6-[4-methoxy-3-(pyridin-2- | 0.008 | 0.051 |
| | ylethynyl)benzoyl]-5,6,7,8-tetrahydro-1,6- | | |
| | naphthyridine | | |
| 15 | 6-[4-methoxy-3-(pyridin-2-ylethynyl)benzoyl]- | 0.096 | NT |
| | 5,6,7,8-tetrahydropyrido[4,3-d]pyrimidin-4(3H)- | | |
| | one | | |
| 16 | 2-[4-methoxy-3-(pyridin-2-ylethynyl)benzoyl]-7- | 0.001 | 0.125 |
| | (trifluoromethyl)-1,2,3,4-tetrahydroisoquinoline | | |
| 17 | 1-{2-[4-methoxy-3-(pyridin-2-ylethynyl)benzoyl]- | 0.612 | NT |
| | 2,3-dihydro-1H-isoindol-1-yl}-2-methylpropan-2-ol | | |
| 18 | 9-[4-methoxy-3-(pyridin-2-ylethynyl)benzoyl]- | 1.124 | NT |
| | 1,2,3,4-tetrahydro-1,4-epiminonaphthalene | | |
| 19 | 5,8-dimethoxy-2-[4-methoxy-3-(pyridin-2- | 0.006 | 0.03 |
| | ylethynyl)benzoyl]-1,2,3,4-tetrahydroisoquinoline | | |
| 20 | 6,7-dimethoxy-2-[4-methoxy-3-(pyridin-2- | 0.036 | 0.049 |
| | ylethynyl)benzoyl]-1,2,3,4-tetrahydroisoquinoline | | |
| 21 | 8-methoxy-2-[4-methoxy-3-(pyridin-2- | 0.003 | 0.034 |
| | ylethynyl)benzoyl]-1,2,3,4-tetrahydroisoquinoline | | |
| 22 | 2-[4-methoxy-3-(pyridin-2-ylethynyl)benzoyl]-1-(2- | 0.022 | 0.22 |
| | thienyl)-1,2,3,4-tetrahydroisoquinoline | | |
| 23 | 2-[4-methoxy-3-(pyridin-2-ylethynyl)benzoyl]-8-[1- | >10 | NT |
| | (methylsulfonyl)piperidin-4-yl]-1,2,3,4- | | |
| | tetrahydroisoquinoline | | |
| 24 | 2-[4-methoxy-3-(pyridin-2-ylethynyl)benzoyl]-4-[4- | 0.52 | NT |
| | (trifluoromethyl)phenoxy]-1,2,3,4- | | |
| | tetrahydroisoquinoline | | |
| 25 | 4-[(1,1-dioxidothiomorpholin-4-yl)sulfonyl]-2-[4- | 0.07 | 0.097 |
| | methoxy-3-(pyridin-2-ylethynyl)benzoyl]-1,2,3,4- | | |
| | tetrahydroisoquinoline | | |
| 26 | 2-[4-methoxy-3-(pyridin-2-ylethynyl)benzoyl]-1- | 0.057 | 0.169 |
| | phenyl-1,2,3,4-tetrahydroisoquinoline | | |
| 27 | 1-(2-furyl)-2-[4-methoxy-3-(pyridin-2- | 0.021 | 0.04 |
| | ylethynyl)benzoyl]-1,2,3,4-tetrahydroisoquinoline | | |
| 28 | (1S)-2-[4-methoxy-3-(pyridin-2-ylethynyl)benzoyl]- | 0.178 | NT |
| | 1-[(2S)-tetrahydrofuran-2-yl]-1,2,3,4- | | |
| | tetrahydroisoquinoline | | |
| 29 | N-({2-[4-methoxy-3-(pyridin-2-ylethynyl)benzoyl]- | 0.068 | 0.447 |
| | 1,2,3,4-tetrahydroisoquinolin-4-yl}methyl)aniline | | |
| 30 | 1-benzyl-2-[4-methoxy-3-(pyridin-2- | 0.327 | NT |
| | ylethynyl)benzoyl]-1,2,3,4-tetrahydroisoquinoline | | |
| 31 | 6-methoxy-2-[4-methoxy-3-(pyridin-2- | 0.008 | 0.061 |
| | ylethynyl)benzoyl]-1,2,3,4-tetrahydroisoquinoline | | |
| 32 | 2-[4-methoxy-3-(pyridin-2-ylethynyl)benzoyl]-5- | 0.003 | 0.042 |
| | methylisoindoline | | |
| 33 | 5-bromo-2-[4-methoxy-3-(pyridin-2- | 0.002 | 0.173 |
| | ylethynyl)benzoyl]isoindoline | | |
| 34 | 5,6-dichloro-2-[4-methoxy-3-(pyridin-2- | 0.017 | 1.449 |
| | ylethynyl)benzoyl]isoindoline | | |
| 35 | 2-[4-methoxy-3-(pyridin-2-ylethynyl)benzoyl]-5- | 0.023 | 0.061 |
| | nitroisoindoline | | |
| 36 | 4-fluoro-2-[4-methoxy-3-(pyridin-2- | 0.006 | 0.025 |
| | ylethynyl)benzoyl]isoindoline | | |
| 37 | 5-fluoro-2-[4-methoxy-3-(pyridin-2- | 0.006 | 0.04 |
| | ylethynyl)benzoyl]isoindoline | | |
| 38 | 4-chloro-2-[4-methoxy-3-(pyridin-2- | 0.002 | 0.051 |
| | ylethynyl)benzoyl]isoindoline | | |
| 39 | 6-[4-methoxy-3-(pyridin-2-ylethynyl)benzoyl]- | 0.023 | 0.026 |
| | 5,6,7,8-tetrahydro-1,6-naphthyridine | | |
| 40 | 6-[4-methoxy-3-(pyridin-2-ylethynyl)benzoyl]-3- | 0.022 | 0.021 |
| | methyl-5,6,7,8-tetrahydro-1,6-naphthyridine | | |
| 41 | 2-[4-methoxy-3-(pyridin-2-ylethynyl)benzoyl]-6- | 0.026 | 0.012 |
| | methyl-2,3-dihydro-1H-pyrrolo[3,4-c]pyridine | | |
| 42 | 2-[4-methoxy-3-(pyridin-2-ylethynyl)benzoyl]-6,7- | 0.02 | 0.028 |
| | dimethyl-2,3-dihydro-1H-pyrrolo[3,4-c]pyridine | | |
| 43 | 7-chloro-2-[4-methoxy-3-(pyridin-2- | 0.012 | 0.017 |
| | ylethynyl)benzoyl]-6-methyl-2,3-dihydro-1H- | | |
| | pyrrolo[3,4-c]pyridine | | |
| 44 | 2-[4-methoxy-3-(pyridin-2-ylethynyl)benzoyl]-6- | 0.02 | 0.014 |
| | methyl-2,3-dihydro-1H-pyrrolo[3,4-c]pyridin-7- | | |
| | amine | | |
| 45 | 7-methoxy-2-[4-methoxy-3-(pyridin-2- | 0.031 | 0.035 |
| | ylethynyl)benzoyl]-6-methyl-2,3-dihydro-1H- | | |
| | pyrrolo[3,4-c]pyridine | | |
| 46 | 2-[4-methoxy-3-(pyridin-2-ylethynyl)benzoyl]-2,3- | 0.031 | 0.018 |
| | dihydro-1H-pyrrolo[3,4-c]pyridine | | |
| 47 | 5-{[4-methoxy-3-(pyridin-2- | 0.03 | 0.033 |
| | ylethynyl)phenyl]carbonyl}-2-methyl-4,5,6,7- | | |
| | tetrahydro[1,3]oxazolo[4,5-c]pyridine | | |
| 48 | 5-{[4-methoxy-3-(pyridin-2- | 0.023 | 0.042 |
| | ylethynyl)phenyl]carbonyl}-2-methyl-4,5,6,7- | | |
| | tetrahydro[1,3]thiazolo[5,4-c]pyridine | | |
| 49 | 7-{[4-methoxy-3-(pyridin-2- | 0.269 | NT |
| | ylethynyl)phenyl]carbonyl}-3-(trifluoromethyl)- | | |
| | 5,6,7,8-tetrahydro[1,2,4]triazolo[4,3-a]pyrazine | | |
| 50 | 7-{[4-methoxy-3-(pyridin-2- | 0.017 | 0.017 |
| | ylethynyl)phenyl]carbonyl}-5,6,7,8-tetrahydro-1,7- | | |
| | naphthyridine | | |
| 51 | 3-chloro-6-{[4-methoxy-3-(pyridin-2- | 0.013 | 0.03 |
| | ylethynyl)phenyl]carbonyl}-5,6,7,8-tetrahydro-1,6- | | |
| | naphthyridine | | |
| 52 | 6-{[4-methoxy-3-(pyridin-2- | 0.029 | 0.049 |
| | ylethynyl)phenyl]carbonyl}-3-(trifluoromethyl)- | | |
| | 5,6,7,8-tetrahydro-1,6-naphthyridine | | |
| 53 | 5-{[4-methoxy-3-(pyridin-2- | 0.027 | 0.004 |
| | ylethynyl)phenyl]carbonyl}-4,5,6,7-tetrahydro-1H- | | |
| | pyrazolo[4,3-c]pyridine | | |
| 54 | 6-{[4-methoxy-3-(pyridin-2- | 0.026 | 0.008 |
| | ylethynyl)phenyl]carbonyl}-6,7-dihydro-5H- | | |
| | pyrrolo[3,4-b]pyridine | | |
| 55 | 4-{[4-methoxy-3-(pyridin-2- | 0.029 | 0.012 |
| | ylethynyl)phenyl]carbonyl}-2,3,4,5-tetrahydro-1,4- | | |
| | benzoxazepine | | |
| 56 | 4-{[4-methoxy-3-(pyridin-2- | 0.112 | NT |
| | ylethynyl)phenyl]carbonyl}-2,3,4,5-tetrahydro-1H- | | |
| | 1,4-benzodiazepine | | |
| 57 | 3-{[4-methoxy-3-(pyridin-2- | 0.015 | 0.017 |
| | ylethynyl)phenyl]carbonyl}-2,3,4,5-tetrahydro-1H- | | |
| | 3-benzazepine | | |
| 58 | 5-{[4-methoxy-3-(pyridin-2- | 0.057 | 0.018 |
| | ylethynyl)phenyl]carbonyl}-4,5,6,7-tetrahydro-1H- | | |
| | imidazo[4,5-c]pyridine | | |
| 59 | 2-{[4-methoxy-3-(pyridin-2- | 0.042 | 0.029 |
| | ylethynyl)phenyl]carbonyl}-2,3,4,5-tetrahydro-1H- | | |
| | 2-benzazepine | | |
| 60 | 2-[4-chloro-3-(pyridin-2- | 0.001 | 0.006 |
| | ylethynyl)benzoyl]isoindoline | | |
| 61 | 2-[4-chloro-3-(pyridin-2-ylethynyl)benzoyl]-1,2,3,4- | 0.0004 | 0.013 |
| | tetrahydroisoquinoline | | |
| 62 | 7-{[4-chloro-3-(pyridin-2- | 0.005 | 0.009 |
| | ylethynyl)phenyl]carbonyl}-5,6,7,8-tetrahydro-1,7- | | |
| | naphthyridine | | |
| 63 | 2-{[4-chloro-3-(pyridin-2- | 0.0002 | 0.014 |
| | ylethynyl)phenyl]carbonyl}-5,8-difluoro-1,2,3,4- | | |
| | tetrahydroisoquinoline | | |
| 64 | 2-{[4-chloro-3-(pyridin-2- | 0.0004 | 0.016 |
| | ylethynyl)phenyl]carbonyl}-5-fluoro-1,2,3,4- | | |
| | tetrahydroisoquinoline | | |
| 65 | 6-{[4-chloro-3-(pyridin-2- | 0.003 | 0.018 |
| | ylethynyl)phenyl]carbonyl}-6,7-dihydro-5H- | | |
| | pyrrolo[3,4-b]pyridine | | |
| 66 | 5-{[4-chloro-3-(pyridin-2- | 0.004 | 0.011 |
| | ylethynyl)phenyl]carbonyl}-4,5,6,7-tetrahydro-1H- | | |
| | pyrazolo[4,3-c]pyridine | | |
| 67 | 5-{[4-chloro-3-(pyridin-2- | 0.016 | 0.027 |
| | ylethynyl)phenyl]carbonyl}-4,5,6,7-tetrahydro-1H- | | |
| | imidazo[4,5-c]pyridine | | |
| 68 | 6-{[4-chloro-3-(pyridin-2- | 0.004 | 0.026 |
| | ylethynyl)phenyl]carbonyl}-5,6,7,8-tetrahydro-1,6- | | |
| | naphthyridine | | |
| 69 | 5-chloro-2-{[4-chloro-3-(pyridin-2- | 0.0001 | 0.134 |
| | ylethynyl)phenyl]carbonyl}-1,2,3,4- | | |
| | tetrahydroisoquinoline | | |
| 70 | 2-{[4-chloro-3-(pyridin-2- | 0.0004 | 0.037 |
| | ylethynyl)phenyl]carbonyl}-7-fluoro-1,2,3,4- | | |
| | tetrahydroisoquinoline | | |
| 71 | 2-[4-fluoro-3-(pyridin-2-ylethynyl)benzoyl]-1,2,3,4- | 0.005 | 0.067 |
| | tetrahydroisoquinoline | | |
| 72 | 7-chloro-2-[4-fluoro-3-(pyridin-2- | 0.002 | 0.029 |
| | ylethynyl)benzoyl]-1,2,3,4-tetrahydroisoquinoline | | |
| 73 | 5-chloro-2-[4-fluoro-3-(pyridin-2- | 0.001 | 0.04 |
| | ylethynyl)benzoyl]-1,2,3,4-tetrahydroisoquinoline | | |
| 74 | 2-[4-fluoro-3-(pyridin-2- | 0.011 | 0.062 |
| | ylethynyl)benzoyl]isoindoline | | |
| 75 | 2-[4-ethoxy-3-(pyridin-2-ylethynyl)benzoyl]- | 0.003 | 0.011 |
| | 1,2,3,4-tetrahydroisoquinoline | | |
| 76 | 7-chloro-2-[4-ethoxy-3-(pyridin-2- | 0.001 | 0.042 |
| | ylethynyl)benzoyl]-1,2,3,4-tetrahydroisoquinoline | | |
| 77 | 5-chloro-2-[4-ethoxy-3-(pyridin-2- | 0.001 | 0.041 |
| | ylethynyl)benzoyl]-1,2,3,4-tetrahydroisoquinoline | | |
| 78 | 2-[4-ethoxy-3-(pyridin-2- | 0.005 | 0.012 |
| | ylethynyl)benzoyl]isoindoline | | |
| 79 | 2-{[4-(cyclopropylmethoxy)-3-(pyridin-2- | 0.023 | 0.034 |
| | ylethynyl)phenyl]carbonyl}-1,2,3,4- | | |
| | tetrahydroisoquinoline | | |
| 80 | 2-{[4-(cyclopropylmethoxy)-3-(pyridin-2- | 0.189 | NT |
| | ylethynyl)phenyl]carbonyl}-2,3-dihydro-1H- | | |
| | isoindole | | |
| 81 | 2-[4-methyl-3-(pyridin-2- | 0.001 | 0.026 |
| | ylethynyl)benzoyl]isoindoline | | |
| 82 | 2-[4-methyl-3-(pyridin-2-ylethynyl)benzoyl]- | 0.001 | 0.027 |
| | 1,2,3,4-tetrahydroisoquinoline | | |
| 83 | 7-chloro-2-[4-methyl-3-(pyridin-2- | 0.0003 | 0.03 |
| | ylethynyl)benzoyl]-1,2,3,4-tetrahydroisoquinoline | | |
| 84 | 5-chloro-2-[4-methyl-3-(pyridin-2- | 0.0003 | 0.048 |
| | ylethynyl)benzoyl]-1,2,3,4-tetrahydroisoquinoline | | |
| 85 | 6-{[4-methyl-3-(pyridin-2- | 0.004 | 0.006 |
| | ylethynyl)phenyl]carbonyl}-5,6,7,8-tetrahydro-1,6- | | |
| | naphthyridine | | |
| 86 | 7-{[4-methyl-3-(pyridin-2- | 0.011 | 0.008 |
| | ylethynyl)phenyl]carbonyl}-5,6,7,8-tetrahydro-1,7- | | |
| | naphthyridine | | |
| 87 | 5-{[4-methyl-3-(pyridin-2- | 0.014 | 0.002 |
| | ylethynyl)phenyl]carbonyl}-4,5,6,7-tetrahydro-3H- | | |
| | imidazo[4,5-c]pyridine | | |
| 88 | 5-{[4-methyl-3-(pyridin-2- | 0.006 | 0.004 |
| | ylethynyl)phenyl]carbonyl}-4,5,6,7-tetrahydro-1H- | | |
| | pyrazolo[4,3-c]pyridine | | |
| 89 | 2-methyl-5-{[4-methyl-3-(pyridin-2- | 0.004 | 0.004 |
| | ylethynyl)phenyl]carbonyl}-4,5,6,7- | | |
| | tetrahydro[1,3]thiazolo[5,4-c]pyridine | | |
| 90 | 2-methyl-5-{[4-methyl-3-(pyridin-2- | 0.016 | 0.009 |
| | ylethynyl)phenyl]carbonyl}-4,5,6,7- | | |
| | tetrahydro[1,3]oxazolo[4,5-c]pyridine | | |
| 91 | 6-{[4-methyl-3-(pyridin-2- | 0.006 | 0.004 |
| | ylethynyl)phenyl]carbonyl}-6,7-dihydro-5H- | | |
| | pyrrolo[3,4-b]pyridine | | |
| 92 | 6-{[3-(pyridin-2-ylethynyl)-4- | 0.003 | 0.007 |
| | (trifluoromethyl)phenyl]carbonyl}-6,7-dihydro-5H- | | |
| | pyrrolo[3,4-b]pyridine | | |
| 93 | 2-{[3-(pyridin-2-ylethynyl)-4- | 0.002 | 0.042 |
| | (trifluoromethyl)phenyl]carbonyl}-1,2,3,4- | | |
| | tetrahydroisoquinoline | | |
| 94 | 7-chloro-2-{[3-(pyridin-2-ylethynyl)-4- | 0.002 | 0.152 |
| | (trifluoromethyl)phenyl]carbonyl}-1,2,3,4- | | |
| | tetrahydroisoquinoline | | |
| 95 | 5-chloro-2-{[3-(pyridin-2-ylethynyl)-4- | 0.004 | 0.163 |
| | (trifluoromethyl)phenyl]carbonyl}-1,2,3,4- | | |
| | tetrahydroisoquinoline | | |
| 96 | 2-{[3-(pyridin-2-ylethynyl)-4- | 0.005 | 0.069 |
| | (trifluoromethyl)phenyl]carbonyl}-2,3-dihydro-1H- | | |
| | isoindole | | |
| 97 | 3-bromo-6-{[3-(pyridin-2-ylethynyl)-4- | 0.001 | 0.013 |
| | (trifluoromethyl)phenyl]carbonyl}-5,6,7,8- | | |
| | tetrahydro-1,6-naphthyridine | | |
| 98 | 6-{[3-(pyridin-2-ylethynyl)-4- | 0.004 | 0.007 |
| | (trifluoromethyl)phenyl]carbonyl}-5,6,7,8- | | |
| | tetrahydro-1,6-naphthyridine | | |
| 99 | 7-{[3-(pyridin-2-ylethynyl)-4- | 0.034 | NT |
| | (trifluoromethyl)phenyl]carbonyl}-5,6,7,8- | | |
| | tetrahydro-1,7-naphthyridine | | |
| 100 | 5-{[3-(pyridin-2-ylethynyl)-4- | 0.004 | 0.005 |
| | (trifluoromethyl)phenyl]carbonyl}-4,5,6,7- | | |
| | tetrahydro-1H-pyrazolo[4,3-c]pyridine | | |
| 101 | 5-{[3-(pyridin-2-ylethynyl)-4- | 0.036 | 0.018 |
| | (trifluoromethyl)phenyl]carbonyl}-4,5,6,7- | | |
| | tetrahydro-1H-imidazo[4,5-c]pyridine | | |
| 102 | 2-{[3-(pyridin-2-ylethynyl)-4- | 0.12 | NT |
| | (trifluoromethyl)phenyl]carbonyl}-2,3-dihydro-1H- | | |
| | pyrrolo[3,4-c]pyridine | | |
| 103 | 2-{4-methoxy-3-[(3- | 0.109 | NT |
| | methoxyphenyl)ethynyl]benzoyl}isoindoline | | |
| 104 | 2-{3-[(2,4-difluorophenyl)ethynyl]-4- | 0.047 | 2.39 |
| | methoxybenzoyl}isoindoline | | |
| 105 | 2-{3-[(4-fluoro-3-methylphenyl)ethynyl]-4- | 0.077 | 2.358 |
| | methoxybenzoyl}isoindoline | | |
| 106 | 2-(4-methoxy-3-{[2- | >10 | NT |
| | (trifluoromethyl)phenyl]ethynyl}benzoyl)isoindoline | | |
| 107 | 2-{4-methoxy-3-[(2- | >10 | NT |
| | methoxyphenyl)ethynyl]benzoyl}isoindoline | | |
| 108 | 2-[3-(cyclohex-1-en-1-ylethynyl)-4- | 0.203 | NT |
| | methoxybenzoyl]isoindoline | | |
| 109 | 2-[4-methoxy-3-(pyridin-3- | 0.3 | NT |
| | ylethynyl)benzoyl]isoindoline | | |
| 110 | 2-{3-[(2-chlorophenyl)ethynyl]-4- | 1.968 | NT |
| | methoxybenzoyl}isoindoline | | |
| 111 | 4-{[5-(1,3-dihydro-2H-isoindol-2-ylcarbonyl)-2- | >10 | NT |
| | methoxyphenyl]ethynyl}benzonitrile | | |
| 112 | 3-{[5-(1,3-dihydro-2H-isoindol-2-ylcarbonyl)-2- | 0.241 | NT |
| | methoxyphenyl]ethynyl}phenol | | |
| 113 | 2-{3-[(3,5-dimethoxyphenyl)ethynyl]-4- | NT | NT |
| | methoxybenzoyl}isoindoline | | |
| 114 | 2-[4-methoxy-3- | 0.003 | 0.566 |
| | (phenylethynyl)benzoyl]isoindoline | | |
| 115 | 2-{4-methoxy-3-[(3- | 0.021 | >10.000 |
| | methylphenyl)ethynyl]benzoyl}isoindoline | | |
| 116 | 2-{3-[(4-fluorophenyl)ethynyl]-4- | 0.249 | NT |
| | methoxybenzoyl}isoindoline | | |
| 117 | 2-{4-methoxy-3-[(4- | 0.027 | 0.62 |
| | methoxyphenyl)ethynyl]benzoyl}isoindoline | | |
| 118 | 2-{3-[(3-chlorophenyl)ethynyl]-4- | 0.008 | 0.54 |
| | methoxybenzoyl}isoindoline | | |
| 119 | 2-{4-methoxy-3-[(6-methylpyridin-2- | 0.007 | 0.094 |
| | yl)ethynyl]benzoyl}isoindoline | | |
| 120 | 2-{4-methoxy-3-[(6-methylpyridin-2- | 0.005 | 0.067 |
| | yl)ethynyl]benzoyl}-1,2,3,4-tetrahydroisoquinoline | | |
| 121 | (4-(difluoromethoxy)-3-(pyridin-2- | 0.006 | 0.001 |
| | ylethynyl)phenyl)(5H-pyrrolo[3,4-b]pyridin-6(7H)- | | |
| | yl)methanone | | |

Variations, modifications, and other implementations of what is described herein will occur to those of ordinary skill in the art without departing from the spirit and the essential characteristics of the present teachings. Accordingly, the invention is intended to include all such modifications and implementations, and their equivalents.

Each reference cited in the present application, including books, patents, published applications, journal articles and other publications, is incorporated herein by reference in its entirety.

1. A compound of Formula I, 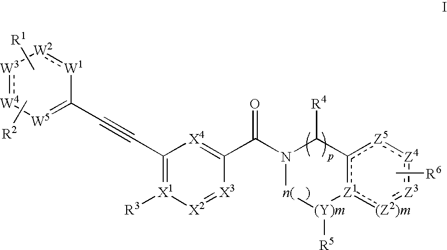
or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and 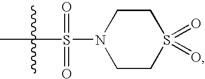
the C₁-C₆alkyl, C₃-C₆cycloalkyl, aryl, heteroaryl, aryloxy, heteroaryloxy, C₁-C₆alkylNR⁷R⁸ and 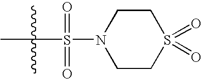
groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S.

2. A compound according to claim 1 or a pharmaceutically acceptable salt thereof, wherein W¹ is selected from N and C.

3. A compound according to claim 2 or a pharmaceutically acceptable salt thereof, wherein W¹ is N.

4. A compound as in claim 1 or a pharmaceutically acceptable salt thereof, wherein Z⁵ is N.

5. A compound as in claim 1 or a pharmaceutically acceptable salt thereof, wherein Z⁴ is N.

6. A compound according to claim 6 or a pharmaceutically acceptable salt thereof, wherein R³ is selected from methoxy, ethoxy, C₁, CF₃, —OCF₃, and —OCHF₂.

7. A compound as in claim 1 or a pharmaceutically acceptable salt thereof, wherein n is 1.

8. A compound as in claim 1 or a pharmaceutically acceptable salt thereof, wherein m is 0.

9. A compound as in claim 1 or a pharmaceutically acceptable salt thereof, wherein p is 1.

10. A compound as in claim 1 or a pharmaceutically acceptable salt thereof, wherein each of R¹, R², R⁴, and R⁵ is H.

11. A compound as in claim 1 or a pharmaceutically acceptable salt thereof, wherein each of W², W³, W⁴, W⁵, X¹, X², X³, X⁴, Z¹, Z², Z³, and Z⁴ is C.

12. A compound as in claim 1 or a pharmaceutically acceptable salt thereof, wherein R⁴, and R⁵ are each H.

13. A compound as in claim 1, wherein the compound selected from the following compounds and pharmaceutically acceptable salts thereof:
2-[4-methoxy-3-(pyridin-2-ylethynyl)benzoyl]isoindoline;
2-[4-methoxy-3-(pyridin-2-ylethynyl)benzoyl]-1,2,3,4-tetrahydroisoquinoline;
5-fluoro-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-5-(trifluoromethyl)-1,2,3,4-tetrahydroisoquinoline;
2-[4-methoxy-3-(pyridin-2-ylethynyl)benzoyl]-8-(trifluoromethyl)-1,2,3,4-tetrahydroisoquinoline;
5,8-difluoro-2-[4-methoxy-3-(pyridin-2-ylethynyl)benzoyl]-1,2,3,4-tetrahydroisoquinoline;
5,7-dichloro-2-[4-methoxy-3-(pyridin-2-ylethynyl)benzoyl]-1,2,3,4-tetrahydroisoquinoline;
7-chloro-2-[4-methoxy-3-(pyridin-2-ylethynyl)benzoyl]-1,2,3,4-tetrahydroisoquinoline;
7-fluoro-2-[4-methoxy-3-(pyridin-2-ylethynyl)benzoyl]-1,2,3,4-tetrahydroisoquinoline;
5-chloro-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1,2,3,4-tetrahydroisoquinoline-7-carbonitrile;
2-[4-methoxy-3-(pyridin-2-ylethynyl)benzoyl]-2,3,4,5-tetrahydro-1H-pyrido[4,3-b]indole;
5-[4-methoxy-3-(pyridin-2-ylethynyl)benzoyl]-4,5,6,7-tetrahydrothieno[3,2-c]pyridine;
3-bromo-6-[4-methoxy-3-(pyridin-2-ylethynyl)benzoyl]-5,6,7,8-tetrahydro-1,6-naphthyridine;
6-[4-methoxy-3-(pyridin-2-ylethynyl)benzoyl]-5,6,7,8-tetrahydropyrido[4,3-d]pyrimidin-4(3H)-one;
2-[4-methoxy-3-(pyridin-2-ylethynyl)benzoyl]-7-(trifluoromethyl)-1,2,3,4-tetrahydroisoquinoline;
1-{2-[4-methoxy-3-(pyridin-2-ylethynyl)benzoyl]-2,3-dihydro-1H-isoindol-1-yl}-2-methylpropan-2-ol;
9-[4-methoxy-3-(pyridin-2-ylethynyl)benzoyl]-1,2,3,4-tetrahydro-1,4-epiminonaphthalene;
5,8-dimethoxy-2-[4-methoxy-3-(pyridin-2-ylethynyl)benzoyl]-1,2,3,4-tetrahydroisoquinoline;
6,7-dimethoxy-2-[4-methoxy-3-(pyridin-2-ylethynyl)benzoyl]-1,2,3,4-tetrahydroisoquinoline;
8-methoxy-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1-(2-thienyl)-1,2,3,4-tetrahydroisoquinoline;
2-[4-methoxy-3-(pyridin-2-ylethynyl)benzoyl]-8-[1-(methylsulfonyl)piperidin-4-yl]-1,2,3,4-tetrahydroisoquinoline;
2-[4-methoxy-3-(pyridin-2-ylethynyl)benzoyl]-4-[4-(trifluoromethyl)phenoxy]-1,2,3,4-tetrahydroisoquinoline;
4-[(1,1-dioxidothiomorpholin-4-yl)sulfonyl]-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-1-phenyl-1,2,3,4-tetrahydroisoquinoline;
1-(2-furyl)-2-[4-methoxy-3-(pyridin-2-ylethynyl)benzoyl]-1,2,3,4-tetrahydroisoquinoline;
(1S)-2-[4-methoxy-3-(pyridin-2-ylethynyl)benzoyl]-1-[(2S)-tetrahydrofuran-2-yl]-1,2,3,4-tetrahydroisoquinoline;
N-({2-[4-methoxy-3-(pyridin-2-ylethynyl)benzoyl]-1,2,3,4-tetrahydroisoquinolin-4-yl}methyl)aniline;
1-benzyl-2-[4-methoxy-3-(pyridin-2-ylethynyl)benzoyl]-1,2,3,4-tetrahydroisoquinoline;
6-methoxy-2-[4-methoxy-3-(pyridin-2-ylethynyl)benzoyl]-1,2,3,4-tetrahydroisoquinoline;
2-[4-methoxy-3-(pyridin-2-ylethynyl)benzoyl]-5-methylisoindoline;
5-bromo-2-[4-methoxy-3-(pyridin-2-ylethynyl)benzoyl]isoindoline;
5,6-dichloro-2-[4-methoxy-3-(pyridin-2-ylethynyl)benzoyl]isoindoline;
2-[4-methoxy-3-(pyridin-2-ylethynyl)benzoyl]-5-nitroisoindoline;
4-fluoro-2-[4-methoxy-3-(pyridin-2-ylethynyl)benzoyl]isoindoline;
5-fluoro-2-[4-methoxy-3-(pyridin-2-ylethynyl)benzoyl]isoindoline;
4-chloro-2-[4-methoxy-3-(pyridin-2-ylethynyl)benzoyl]isoindoline;
6-[4-methoxy-3-(pyridin-2-ylethynyl)benzoyl]-5,6,7,8-tetrahydro-1,6-naphthyridine;
6-[4-methoxy-3-(pyridin-2-ylethynyl)benzoyl]-3-methyl-5,6,7,8-tetrahydro-1,6-naphthyridine;
2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-6,7-dimethyl-2,3-dihydro-1H-pyrrolo[3,4-c]pyridine;
7-chloro-2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-6-methyl-2,3-dihydro-1H-pyrrolo[3,4-c]pyridin-7-amine;
7-methoxy-2-[4-methoxy-3-(pyridin-2-ylethynyl)benzoyl]-6-methyl-2,3-dihydro-1H-pyrrolo[3,4-c]pyridine;
2-[4-methoxy-3-(pyridin-2-ylethynyl)benzoyl]-2,3-dihydro-1H-pyrrolo[3,4-c]pyridine;
5-{[4-methoxy-3-(pyridin-2-ylethynyl)phenyl]carbonyl}-2-methyl-4,5,6,7-tetrahydro[1,3]oxazolo[4,5-c]pyridine;
5-{[4-methoxy-3-(pyridin-2-ylethynyl)phenyl]carbonyl}-2-methyl-4,5,6,7-tetrahydro[1,3]thiazolo[5,4-c]pyridine;
7-{[4-methoxy-3-(pyridin-2-ylethynyl)phenyl]carbonyl}-3-(trifluoromethyl)-5,6,7,8-tetrahydro[1, 2,4]triazolo[4,3-a]pyrazine;
7-{[4-methoxy-3-(pyridin-2-ylethynyl)phenyl]carbonyl}-5,6,7,8-tetrahydro-1,7-naphthyridine;
3-chloro-6-{[4-methoxy-3-(pyridin-2-ylethynyl)phenyl]carbonyl}-5,6,7,8-tetrahydro-1,6-naphthyridine;
6-{[4-methoxy-3-(pyridin-2-ylethynyl)phenyl]carbonyl}-3-(trifluoromethyl)-5,6,7,8-tetrahydro-1,6-naphthyridine;
5-{[4-methoxy-3-(pyridin-2-ylethynyl)phenyl]carbonyl}-4,5,6,7-tetrahydro-1H-pyrazolo[4,3-c]pyridine;
6-{[4-methoxy-3-(pyridin-2-ylethynyl)phenyl]carbonyl}-6,7-dihydro-5H-pyrrolo[3,4-b]pyridine;
4-{[4-methoxy-3-(pyridin-2-ylethynyl)phenyl]carbonyl}-2,3,4,5-tetrahydro-1,4-benzoxazepine;
4-{[4-methoxy-3-(pyridin-2-ylethynyl)phenyl]carbonyl}-2,3,4,5-tetrahydro-1H-1,4-benzodiazepine;
3-{[4-methoxy-3-(pyridin-2-ylethynyl)phenyl]carbonyl}-2,3,4,5-tetrahydro-1H-3-benzazepine;
5-{[4-methoxy-3-(pyridin-2-ylethynyl)phenyl]carbonyl}-4,5,6,7-tetrahydro-1H-imidazo[4,5-c]pyridine;
2-{[4-methoxy-3-(pyridin-2-ylethynyl)phenyl]carbonyl}-2,3,4,5-tetrahydro-1H-2-benzazepine;
2-[4-chloro-3-(pyridin-2-ylethynyl)benzoyl]isoindoline;
2-[4-chloro-3-(pyridin-2-ylethynyl)benzoyl]-1,2,3,4-tetrahydroisoquinoline;
7-{[4-chloro-3-(pyridin-2-ylethynyl)phenyl]carbonyl}-5,6,7,8-tetrahydro-1,7-naphthyridine;
2-{[4-chloro-3-(pyridin-2-ylethynyl)phenyl]carbonyl}-5,8-difluoro-1,2,3,4-tetrahydroisoquinoline;
2-{[4-chloro-3-(pyridin-2-ylethynyl)phenyl]carbonyl}-5-fluoro-1,2,3,4-tetrahydroisoquinoline;
6-{[4-chloro-3-(pyridin-2-ylethynyl)phenyl]carbonyl}-6,7-dihydro-5H-pyrrolo[3,4-b]pyridine;
5-{[4-chloro-3-(pyridin-2-ylethynyl)phenyl]carbonyl}-4,5,6,7-tetrahydro-1H-pyrazolo[4,3-c]pyridine;
5-{[4-chloro-3-(pyridin-2-ylethynyl)phenyl]carbonyl}-4,5,6,7-tetrahydro-1H-imidazo[4,5-c]pyridine;
6-{[4-chloro-3-(pyridin-2-ylethynyl)phenyl]carbonyl}-5,6,7,8-tetrahydro-1,6-naphthyridine;
5-chloro-2-{[4-chloro-3-(pyridin-2-ylethynyl)phenyl]carbonyl}-1,2,3,4-tetrahydroisoquinoline;
2-{[4-chloro-3-(pyridin-2-ylethynyl)phenyl]carbonyl}-7-fluoro-1,2,3,4-tetrahydroisoquinoline;
2-[4-fluoro-3-(pyridin-2-ylethynyl)benzoyl]-1,2,3,4-tetrahydroisoquinoline;
7-chloro-2-[4-fluoro-3-(pyridin-2-ylethynyl)benzoyl]-1,2,3,4-tetrahydroisoquinoline;
5-chloro-2-[4-fluoro-3-(pyridin-2-ylethynyl)benzoyl]-1,2,3,4-tetrahydroisoquinoline;
2-[4-fluoro-3-(pyridin-2-ylethynyl)benzoyl]isoindoline;
2-[4-ethoxy-3-(pyridin-2-ylethynyl)benzoyl]-1,2,3,4-tetrahydroisoquinoline;
7-chloro-2-[4-ethoxy-3-(pyridin-2-ylethynyl)benzoyl]-1,2,3,4-tetrahydroisoquinoline;
5-chloro-2-[4-ethoxy-3-(pyridin-2-ylethynyl)benzoyl]-1,2,3,4-tetrahydroisoquinoline;
2-[4-ethoxy-3-(pyridin-2-ylethynyl)benzoyl]isoindoline;
2-{[4-(cyclopropylmethoxy)-3-(pyridin-2-ylethynyl)phenyl]carbonyl}-1,2,3,4-tetrahydroisoquinoline;
2-{[4-(cyclopropylmethoxy)-3-(pyridin-2-ylethynyl)phenyl]carbonyl}-2,3-dihydro-1H-isoindole;
2-[4-methyl-3-(pyridin-2-ylethynyl)benzoyl]isoindoline;
2-[4-methyl-3-(pyridin-2-ylethynyl)benzoyl]-1,2,3,4-tetrahydroisoquinoline;
7-chloro-2-[4-methyl-3-(pyridin-2-ylethynyl)benzoyl]-1,2,3,4-tetrahydroisoquinoline;
5-chloro-2-[4-methyl-3-(pyridin-2-ylethynyl)benzoyl]-1,2,3,4-tetrahydroisoquinoline;
6-{[4-methyl-3-(pyridin-2-ylethynyl)phenyl]carbonyl}-5,6,7,8-tetrahydro-1,6-naphthyridine;
7-{[4-methyl-3-(pyridin-2-ylethynyl)phenyl]carbonyl}-5,6,7,8-tetrahydro-1,7-naphthyridine;
5-{[4-methyl-3-(pyridin-2-ylethynyl)phenyl]carbonyl}-4,5,6,7-tetrahydro-3H-imidazo[4,5-c]pyridine;
5-{[4-methyl-3-(pyridin-2-ylethynyl)phenyl]carbonyl}-4,5,6,7-tetrahydro-1H-pyrazolo[4,3-c]pyridine;
2-methyl-5-{[4-methyl-3-(pyridin-2-ylethynyl)phenyl]carbonyl}-4,5,6,7-tetrahydro[1,3]thiazolo[5,4-c]pyridine;
2-methyl-5-{[4-methyl-3-(pyridin-2-ylethynyl)phenyl]carbonyl}-4,5,6,7-tetrahydro[1,3]oxazolo[4,5-c]pyridine;
6-{[4-methyl-3-(pyridin-2-ylethynyl)phenyl]carbonyl}-6,7-dihydro-5H-pyrrolo[3,4-b]pyridine;
6-{[3-(pyridin-2-ylethynyl)-4-(trifluoromethyl)phenyl]carbonyl}-6,7-dihydro-5H-pyrrolo[3,4-b]pyridine;
2-{[3-(pyridin-2-ylethynyl)-4-(trifluoromethyl)phenyl]carbonyl}-1,2,3,4-tetrahydroisoquinoline;
7-chloro-2-{[3-(pyridin-2-ylethynyl)-4-(trifluoromethyl)phenyl]carbonyl}-1,2,3,4-tetrahydroisoquinoline;
5-chloro-2-{[3-(pyridin-2-ylethynyl)-4-(trifluoromethyl)phenyl]carbonyl}-1,2,3,4-tetrahydroisoquinoline;
2-{[3-(pyridin-2-ylethynyl)-4-(trifluoromethyl)phenyl]carbonyl}-2,3-dihydro-1H-isoindole;
3-bromo-6-{[3-(pyridin-2-ylethynyl)-4-(trifluoromethyl)phenyl]carbonyl}-5,6,7,8-tetrahydro-1,6-naphthyridine;
6-{[3-(pyridin-2-ylethynyl)-4-(trifluoromethyl)phenyl]carbonyl}-5,6,7,8-tetrahydro-1,6-naphthyridine;
7-{[3-(pyridin-2-ylethynyl)-4-(trifluoromethyl)phenyl]carbonyl}-5,6,7,8-tetrahydro-1,7-naphthyridine;
5-{[3-(pyridin-2-ylethynyl)-4-(trifluoromethyl)phenyl]carbonyl}-4,5,6,7-tetrahydro-1H-pyrazolo[4,3-c]pyridine;
5-{[3-(pyridin-2-ylethynyl)-4-(trifluoromethyl)phenyl]carbonyl}-4,5,6,7-tetrahydro-1H-imidazo[4,5-c]pyridine;
2-{[3-(pyridin-2-ylethynyl)-4-(trifluoromethyl)phenyl]carbonyl}-2,3-dihydro-1H-pyrrolo[3,4-c]pyridine;
2-{4-methoxy-3-[(3-methoxyphenyl)ethynyl]benzoyl}isoindoline;
2-{3-[(2,4-difluorophenyl)ethynyl]-4-methoxybenzoyl}isoindoline;
2-{3-[(4-fluoro-3-methylphenyl)ethynyl]-4-methoxybenzoyl}isoindoline;
2-(4-methoxy-3-{[2-(trifluoromethyl)phenyl]ethynyl}benzoyl)isoindoline;
2-{4-methoxy-3-[(2-methoxyphenyl)ethynyl]benzoyl}isoindoline;
2-[3-(cyclohex-1-en-1-ylethynyl)-4-methoxybenzoyl]isoindoline;
2-[4-methoxy-3-(pyridin-3-ylethynyl)benzoyl]isoindoline;
2-{3-[(2-chlorophenyl)ethynyl]-4-methoxybenzoyl}isoindoline;
4-{[5-(1,3-dihydro-2H-isoindol-2-ylcarbonyl)-2-methoxyphenyl]ethynyl}benzonitrile;
3-{[5-(1,3-dihydro-2H-isoindol-2-ylcarbonyl)-2-methoxyphenyl]ethynyl}phenol;
2-{3-[(3,5-dimethoxyphenyl)ethynyl]-4-methoxybenzoyl}isoindoline;
2-[4-methoxy-3-(phenylethynyl)benzoyl]isoindoline;
2-{4-methoxy-3-[(3-methylphenyl)ethynyl]benzoyl}isoindoline;
2-{3-[(4-fluorophenyl)ethynyl]-4-methoxybenzoyl}isoindoline;
2-{4-methoxy-3-[(4-methoxyphenyl)ethynyl]benzoyl}isoindoline;
2-{3-[(3-chlorophenyl)ethynyl]-4-methoxybenzoyl}isoindoline;
2-[3-(Pyridin-2-ylethynyl)benzoyl]isoindoline;
(4-(difluoromethoxy)-3-(pyridin-2-ylethynyl)phenyl)(5H-pyrrolo[3,4-b]pyridin-6(7H)-yl)methanone;
2-{4-methoxy-3-[(6-methylpyridin-2-yl)ethynyl]benzoyl}isoindoline; and
2-{4-methoxy-3-[(6-methylpyridin-2-yl)ethynyl]benzoyl}-1,2,3,4-tetrahydroisoquinoline.

14. A pharmaceutical composition comprising:
a compound of Formula I, 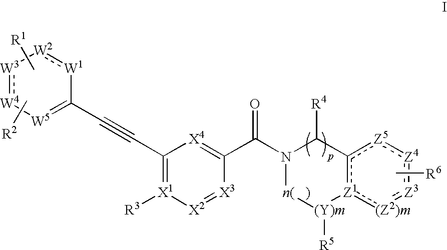
or a pharmaceutically acceptable salt thereof, wherein
W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and 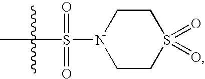
the C₁-C₆alkyl, C₃-C₆cycloalkyl, aryl, heteroaryl, aryloxy, heteroaryloxy, C₁-C₆alkylNR⁷R⁸ and 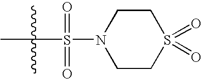
groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino; and
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S; and
a pharmaceutically acceptable excipient.

15. Use of a compound of Formula I, 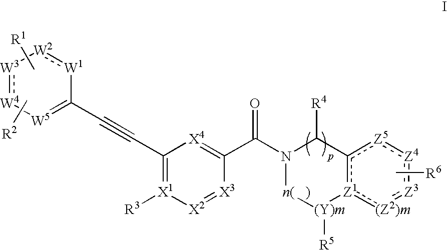
or a pharmaceutically acceptable salt thereof,
wherein W¹, W², W³, W⁴, W⁵, X¹, X², X³, X⁴, Y and Z¹, Z², Z³, Z⁴, Z⁵ are each independently selected from C, N, S, and O; [symbol]
is independently a single bond or a double bond;
n is 1 or 2;
m is 0 or 1;
p is 1 or 2;
R¹ and R² are each independently selected from H, hydroxyl, halogen, cyano, C₁-C₆alkyl, halo-C₁-C₆alkyl, and —O(C₁-C₆alkyl);
R³ is selected from H, halogen, C₁-C₆alkyl, halo-C₁-C₆alkyl, —O(C₁-C₆alkyl), halo-C₁-C₆alkoxy, and hydroxyl;
R⁴ and R⁵ are each independently selected from H, C₁-C₆alkyl, C₃-C₆cycloalkyl, aryl, heteroaryl, aryloxy, heteroaryloxy, C₁-C₆alkylNR⁷R⁸ and 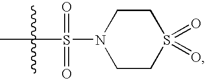
the C₁-C₆alkyl, C₃-C₆cycloalkyl, aryl, heteroaryl, aryloxy, heteroaryloxy, C₁-C₆alkylNR⁷R⁸ and 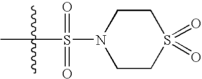
groups are optionally independently substituted with from 1 to 4-C₁-C₆alkyl, halo-C₁-C₆alkyl, —OH, or —NH₂; or R⁴ and R⁵ and the atoms to which they are attached form a ring;
R⁶ is from 1 to 4 groups each independently selected from H, halogen, oxo, cyano, C₁-C₆alkyl, halo-C₁-C₆alkyl, —O(C₁-C₆alkyl), heterocyclyl, NO₂, and amino;
R⁷ and R⁸ are each independently selected from H, C₁-C₆alkyl, aryl and R⁷ and R⁸ can be taken together with the nitrogen to which they are attached to form a saturated heterocycle containing 5-7 atoms independently selected from C, N, O, or S; for the preparation of a medicament for the treatment of a disorder or condition selected from depression, anxiety, panic disorders, social anxiety, obsessive compulsive disorders, generalized anxiety disorders, phobias, post-traumatic stress disorder, bipolar disorder, Asperger's syndrome, pervasive developmental disorders, gastrointestinal disorders such as gastroesophageal reflux disease, dyspepsia, irritable bowel syndrome, functional bloating, functional diarrhea, chronic constipation, functional disturbances of the biliary tract, migraine, chronic pain, fibromyalgia, neuropathic pain, post-herpetic neuropathic pain, addiction, Parkinson's disease, senile dementia, levadopa-induced dyskinesia, Alzheimer's disease, Huntington's chorea, amyotrophic lateral sclerosis, multiple sclerosis, Down Syndrome, fragile-X syndrome, autistic spectrum disorders, attention deficit hyperactivity disorder, stroke, ischemic injury, epilepsy, hypoglycemia, obesity or a combination thereof.
